(19) United States
(12) Patent Application Publication
(10) Pub. No.: US 2021/0130413 A1
(43) Pub. Date: May 6, 2021

(54) MODIFIED AAV CAPSIDS AND USES THEREOF

(71) Applicant: Adverum Biotechnologies, Inc., Redwood City, CA (US)

(72) Inventor: Annahita KERAVALA, Palo Alto, CA (US)

(21) Appl. No.: 16/488,689

(22) PCT Filed: Feb. 28, 2018
(86) PCT No.: PCT/US18/20215

§ 371 Date: Aug. 26, 2019

Related U.S. Application Data
(60) Provisional application No. 62/464,878, filed on Feb. 28, 2017.

(51) Int. Cl. C07K 14/005; C12N 15/86; C07K 16/22; C07K 14/81; C07K 14/755; A61K 48/00; A61K 9/00

(52) U.S. Cl. CPC C07K 14/005; C12N 15/86; C07K 16/22; C07K 14/81; C12N 2750/14145; A61K 48/0075; A61K 9/0048; C12N 2750/14122; C12N 2750/14143; C07K 14/755

(57) ABSTRACT

The present disclosure provides modified adeno-associated virus (AAV) virions with altered capsid proteins, where the modified AAV virions exhibit greater infectivity of retinal cells when administered to the eye or greater infectivity of liver cells when administered intravenously. The present disclosure further provides methods of delivering a gene product to a retinal cell in an individual, methods of treating ocular diseases and disorders, methods of delivering a gene product to the liver in an individual, and methods of treating liver diseases and disorders.

### CROSS-REFERENCE TO RELATED APPLICATIONS

This application claims the benefit of U.S. Provisional Application Ser. No. 62/464,878, filed Feb. 28, 2017, the full disclosure of which is herein incorporated by reference.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is AVBI_012_01WO_ST25.txt. The text file is 22 KB, was created on Feb. 27, 2018, and is being submitted electronically via EFS-Web.

### FIELD OF THE INVENTION

Embodiments of the present disclosure related to modified viral capsid proteins, including modified AAV capsid proteins, viruses and viral vectors comprising the modified AAV capsid proteins, and methods of using these viruses and viral vectors to deliver polypeptides to cells.

### BACKGROUND OF THE INVENTION

A promising approach to treating and preventing genetic and other diseases and disorders is delivery of therapeutic agents with a gene therapy vector such as a viral vector. Illustrative examples of viral vectors suitable for gene therapy include but are not limited to retroviral vectors, lentiviral vectors, adenovirus vectors, herpes virus vectors, alphavirus vectors, and adeno-associated virus (AAV) vectors.

AAV is a 4.7 kb, single-stranded DNA virus. Recombinant vectors based on AAV are associated with excellent clinical safety, since wild-type AAV is nonpathogenic and has no etiologic association with any known diseases. In addition, AAV offers the capability for highly efficient gene delivery and sustained transgene expression in numerous tissues, including eye, muscle, lung and brain. Furthermore, AAV has shown promise in human clinical trials, including a Leber's congenital amaurosis trial in which patients treated with a therapeutic delivered by a single subretinal administration of an rAAV vector experienced sustained clinical benefit from expression of the therapeutic agent for more than four years from the initial date of treatment.

Certain challenges that remain with regard to the design of viral vectors for use in gene therapy include optimizing viral cell tropism and tissue specific delivery. Thus, there is a need for optimized vectors for expressing genes in selected mammalian cell types and tissues. The present invention addresses this need by providing modified AAV capsid proteins advantageous for the delivery of viral vectors to desired cells and tissues.

### SUMMARY OF THE INVENTION

The present invention relates generally to the field of gene therapy, and in particular, to viral vectors useful for the delivery of nucleic acid segments encoding various agents (e.g., peptides, polypeptides, ribozymes, and catalytic RNA molecules), including therapeutic agents, to selected cells and tissues of vertebrate animals. In particular, aspects of the present invention include modified capsid proteins that are useful in gene therapy vectors, including for example, herpes simplex virus (HSV), alphavirus (AV), and AAV vectors, for the delivery of agents to desired cells or tissues, e.g., retina or liver, and for the treatment of mammalian diseases, disorders, and dysfunctions. In certain embodiments, the viral vectors of the present invention are variants of ShH10, which is an AAV with a better neutralizing antibody profile than many other AAV serotypes. Thus, variant AAVs of the present invention provide the advantage of retaining this good neutralizing antibody profile while having a different tropism, e.g., altered heparan sulfate binding, retina specific tropism, or liver specific tropism.

The disclosed compositions may be utilized in a variety of investigative, diagnostic and therapeutic regimens, including the prevention and treatment of a variety of human diseases.

In certain embodiments, the present invention includes a non-naturally-occurring modified AAV capsid protein, comprising one or more amino acid modifications. In certain embodiments, the modified AAV capsid protein comprises an amino acid or peptide insertion comprising or consisting of an amino acid sequence having at least 80%, at least 85%, or at least 90% homology to the amino acid sequence, LGETTRP (SEQ ID NO:6). In some embodiments, the modified AAV capsid protein comprises an amino acid or peptide insertion comprising or consisting of an amino acid sequence having at least 80%, at least 85%, or at least 90% homology to the amino acid sequence LALGETTRPA (SEQ ID NO:14), or a fragment of the amino acid sequence comprising at least five, at least six, at least seven, at least eight, or at least nine consecutive amino acids thereof, which insertion is referred to as a 7m8 amino acid insertion. In certain embodiments, the AAV is AAVShH10, and the 7m8 amino acid insertion is located between amino acid residues 456 and 457, between amino acid residues 457 and 458, or between amino acid residues 458 and 459 of the AAVShH10 capsid protein. Unless otherwise indicated, the capsid protein amino acid sequences referred to herein are VP1 sequences. A skilled artisan will understand that equivalent sequences exist in VP2 and VP3 capsid protein amino acid sequences, and the present disclosure also includes modified VP2 and VP3 capsid proteins having any of the modifications, e.g., insertions, described herein. In certain embodiments, the AAV is a different AAV, such as, e.g., AAV1, AAV2, AAV6, AAV8, AAV9 or AAV10, and the 7m8 amino acid insertion is located in the capsid proteins of these other AAVs between amino acid residues corresponding to amino acid residues 456 and 457, amino acid residues 457 and 458, or amino acid residues 458 and 459 of the AAVShH10 capsid protein. It is understood that the corresponding residues in other AAVs may have different amino acid numbers. The skilled artisan could determine these residues based on sequence alignments, crystal structure, and the location of heparan sulfate proteoglycan binding sites. In particular embodiments, the insertion is close to but does not completely disrupt the HSPG binding site's activity. In certain embodiments, it is adjacent to an amino acid residue identified as being important for HSPG binding.

In a related embodiment, the present invention includes a polynucleotide comprising a nucleic acid sequence encoding a modified AAV capsid protein described herein. In certain embodiments, the nucleic acid sequence encoding the modified AAV capsid protein is operably linked to a promoter sequence. In particular embodiments, the polynucleotide further comprises a nucleic acid sequence encoding a rep protein.

A further related embodiment of the present invention includes a cell comprising an expression vector described herein. In further embodiments, the cell comprises a polynucleotide that encodes a therapeutic protein.

Another embodiment is a recombinant virus or viral vector comprising a modified capsid protein described herein. The AAV virion of this invention may exhibit altered affinity for heparan sulfate binding relative to AAVShH10. In particular embodiments, the recombinant virus or viral vector is eluted from a heparan sulfate column at a salt concentration of about 0.2 M to about 0.4 M, e.g., about 0.2 M, about 0.3 M or about 0.4 M. In some embodiments, the recombinant virus or viral vector is capable of binding to and crossing the inner limiting membrane (ILM) when intravitreally injected into a mammal. In certain embodiments, the recombinant virus or viral vector comprises a polynucleotide sequence that encodes a therapeutic protein. In particular embodiments, the therapeutic protein is an anti-vascular endothelial growth factor (anti-VEGF) agent. In particular embodiments, the therapeutic protein is alpha-1 antitrypsin, factor IX, factor VIII, C1-esterase inhibitor, β-globin or γ-globin. In certain embodiments, the therapeutic protein is one that exerts its therapeutic effect when expressed systemically, e.g., wherein the viral vector transduces the liver, which then produces the therapeutic protein, resulting in it being delivered systemically.

In certain embodiments, a recombinant virus or viral vector described herein has an altered cellular tropism as compared to a corresponding virus or viral vector having a wild type capsid protein, i.e., the same capsid protein absent the 7m8 insertion. In some embodiments, the recombinant virus or viral vector has a greater tropism for retinal cells or liver cells. In one embodiment, the recombinant virus or viral vector comprises an AAVShH10 capsid having a 7m8 insert between amino acid residues 456 and 457. In one embodiment, the recombinant virus or viral vector comprises an AAVShH10 capsid having a 7m8 insert between amino acid residues 457 and 458. In one embodiment, the recombinant virus or viral vector comprises an AAVShH10 capsid having a 7m8 insert between amino acid residues 458 and 459.

In further related embodiments, the present invention includes a pharmaceutical composition comprising a recombinant virus or viral vector described herein.

The present invention also includes a related method of providing a protein to a retina of a subject, comprising administering to the eye of the subject, e.g., by intravitreal injection, a recombinant virus or viral vector or pharmaceutical composition described herein, wherein the recombinant virus or viral vector comprises a polynucleotide sequence that encodes the protein. In one embodiment, the recombinant virus or viral vector comprises an AAVShH10 capsid having a 7m8 insert between amino acid residues 456 and 457, amino acid residues 457 and 458, or amino acid residues 458 and 459.

The present invention also includes a related method of providing a protein to the liver of a subject, comprising administering to the subject, e.g., intravenously, a recombinant virus or viral vector or pharmaceutical composition described herein, wherein the recombinant virus or viral vector comprises a polynucleotide sequence that encodes the protein. In one embodiment, the recombinant virus or viral vector comprises an AAVShH10 capsid having a 7m8 insert between amino acid residues 456 and 457, amino acid residues 457 and 458, or amino acid residues 458 and 459.

The present invention further includes a method of providing a therapeutic gene product (e.g., a therapeutic protein) to a retina of a subject in need thereof, comprising administering to the subject, e.g., by intravitreal injection, a pharmaceutical composition comprising a recombinant virus or viral vector described herein, wherein the recombinant virus or viral vector comprises a polynucleotide encoding the therapeutic gene product. In particular embodiments, subject has been diagnosed with or is considered at risk of an ocular disease or disorder. In particular embodiments, the subject has been diagnosed with or is suspected of having or being at risk of developing one or more conditions selected from the group consisting of: age-related macular degeneration (AMD), wet-AMD, dry-AMD, retinal neovascularization, choroidal neovascularization, diabetic retinopathy, proliferative diabetic retinopathy, retinal vein occlusion, central retinal vein occlusion, branched retinal vein occlusion, diabetic macular edema, diabetic retinal ischemia, ischemic retinopathy, and diabetic retinal edema. In one embodiment, the recombinant virus or viral vector comprises an AAVShH10 capsid having a 7m8 insert between amino acid residues 456 and 457, amino acid residues 457 and 458, or amino acid residues 458 and 459.

The present invention further includes a method of providing a therapeutic gene product (e.g., a therapeutic protein) to the liver of a subject in need thereof, comprising administering to the subject, e.g., intravenously, a pharmaceutical composition comprising a recombinant virus or viral vector described herein, wherein the recombinant virus or viral vector comprises a polynucleotide encoding the therapeutic gene product. In particular embodiments, subject has been diagnosed with or is considered at risk of a liver disease or disorder. In particular embodiments, the subject has been diagnosed with or is suspected of having or being at risk of developing one or more conditions selected from the group consisting of: alpha-1 antitrypsin deficiency, hemophilia B, hemophilia A, hereditary angioedema, and β-thalassemia. In one embodiment, the recombinant virus or viral vector comprises an AAVShH10 capsid having a 7m8 insert between amino acid residues 456 and 457, amino acid residues 457 and 458, or amino acid residues 458 and 459.

The present invention further provides a method of altering the tropism of an AAVShH10, AAV1, or AAV6 virus or viral vector, comprising introducing a 7m8 insertion into the capsid protein of the virus or viral vector, e.g., between any of amino acid residues 456 and 457, amino acid residues 457 and 458, or amino acid residues 458 and 459.

### BRIEF DESCRIPTION OF THE DRAWINGS

The novel features of the disclosure are set forth with particularity in the appended claims. A better understanding of the features and advantages of the present invention will be obtained by reference to the following detailed description that sets forth illustrative embodiments, in which the principles of the invention are utilized, and the accompanying drawings.

FIGS. 1A-1F show immunofluorescence images of HEK293 cells either not transduced (FIG. 1A) or transduced with AAV.7m8 (FIG. 1B), ShH10 (FIG. 1C), ShH10/7m8(457) (FIG. 1D), ShH10/7m8(458) (FIG. 1E), or ShH10/7m8(459) (FIG. 1F) virus encoding green fluorescent protein (GFP) at MOI of 3×10⁵.

FIGS. 2A-2F provides graphs showing the results of flow cytometry analysis of HEK293 cells either not transduced (FIG. 2A) or transduced with ShH10 (FIG. 2B), AAV.7m8 (FIG. 2C), ShH10/7m8(457) (FIG. 2D), ShH10/7m8(458) (FIG. 2E) or ShH10/7m8(459) (FIG. 2F) virus encoding green fluorescent protein (GFP).

FIGS. 3A-3C provide data generated from flow cytometry analysis of HEK293 cells either not transduced or transduced with ShH10, AAV.7m8, ShH10/7m8(457), ShH10/7m8(458) or ShH10/7m8(459) virus encoding green fluorescent protein (GFP). FIG. 3A shows average percentage of GFP positive cells for each virus; FIG. 3B shows median fluorescence intensity (MFI) for each virus, and FIG. 3C is a table summarizing the results shown in FIGS. 3A and 3B. Error bars in each graph show standard deviation.

FIGS. 4A-4D show immunofluorescence (left panel) and light microscopy (right panel) images of U87 cells transduced with ShH10 (FIG. 4A), ShH10/7m8(457) (FIG. 4B), ShH10/7m8(458) (FIG. 4C), or ShH10/7m8(459) (FIG. 4D) virus encoding green fluorescent protein (GFP) at MOI of 3×10⁵.

FIGS. 5A-5F provide graphs showing the results of flow cytometry analysis of U87 cells either not transduced (FIG. 5A) or transduced with ShH10 (FIG. 5B), ShH10/7m8(457) (FIG. 5C), ShH10/7m8(458) (FIG. 5D), or ShH10/7m8(459) (FIG. 5E) virus encoding green fluorescent protein (GFP) and a table summarizing the percentage of cells expressing GFP and the MFI for each virus (FIG. 5F).

FIGS. 6A-6D show immunofluorescence (left panel) and light microscopy images (right panel) of HepG2 cells transduced with ShH10 (FIG. 6A), ShH10/7m8(457) (FIG. 6B), ShH10/7m8(458) (FIG. 6C), or ShH10/7m8(459) (FIG. 6D) virus encoding green fluorescent protein (GFP) at MOI of 3×10⁵.

FIGS. 7A-7E provide graphs showing the results of flow cytometry analysis of HepG2 cells transduced with ShH10 (FIG. 7A), ShH10/7m8(457) (FIG. 7B), ShH10/7m8(458) (FIG. 7C), or ShH10/7m8(459) (FIG. 7D), virus encoding green fluorescent protein (GFP) and a table summarizing the percentage of cells expressing GFP and the MFI for each virus (FIG. 7E).

FIGS. 8A-8F outlines a heparin binding assay (FIG. 8A) and shows the dot blot results of heparan sulfate column fractions from AAV.7m8 (FIG. 8B), ShH10 (FIG. 8C), ShH10/7m8(457) (FIG. 8D), ShH10/7m8(458) (FIG. 8E), or ShH10/7m8(459) (FIG. 8F). Eluates E1 to E10 have increasing concentrations of salt of 0.1M (E1), 0.2M (E2), 0.3M (E3), 0.4M (E4), 0.5M (E5), 0.6M (E6), 0.7M (E7), 0.8M (E8), 0.9M (E9) and 1.0M (E10).

FIG. 9 is a graph showing the neutralizing antibody profile of ShH10/7m8(458) vector expressing GFP under control of the CMV promoter. The ShH10/7m8(458) vector was incubated with various dilutions of IVIG prior to transduction of the 293T cells, which were then incubated for 3 days, prior to analyzing GFP expression.

FIG. 10A-10P show immunofluorescence images of retinal cells from pig explants two weeks following infection with ShH10/7m8(457) or parent ShH10 virus expressing GFP at an MOI of 4×10⁴. FIGS. 10A-10D show single-channel immunofluorescence images taken of a pig retinal explant transduced with ShH10/7m8(457) expressing GFP, stained for GFAP (to detect Muller cells) (FIG. 10A), DAPI (to detect cell nuclei) (FIG. 10B), CHX10 (to detect bipolar cells) (FIG. 10C), and GFP (FIG. 10D). FIGS. 10E-10H show single-channel immunofluorescence images taken of a pig retinal explant transduced with ShH10/7m8(457) expressing GFP, stained for Rhodopsin (to detect rod cells) (FIG. 10E), DAPI (to detect cell nuclei) (FIG. 10F), CHX10 (to detect bipolar cells) (FIG. 10G), or GFP (FIG. 10H). FIGS. 10I-10L show single-channel immunofluorescence images taken of a pig retinal explant transduced with ShH10/7m8(457) expressing GFP, stained for TuJ1 (to detect retinal ganglion cells) (FIG. 10I), DAPI (to detect cell nuclei) (FIG. 10J), CHX10 (to detect bipolar cells) (FIG. 10K), and GFP (FIG. 10L). FIGS. 10M-10P show single-channel immunofluorescence images taken of a pig retinal explant transduced with ShH10 expressing GFP, stained with DAPI (to detect cell nuclei) (FIG. 10M), Rhodopsin (to detect rod cells) (FIG. 10N), GFAP (to detect Muller cells) (FIG. 10O), and GFP (FIG. 10P).

FIGS. 11A-11E show images of retinal cells from gerbil retina following transduction with 2×10¹⁰ vg/eye of parent ShH10 or ShH10/7m8(457) virus expressing GFP. FIG. 11A depicts a fluorescent fundus image of a gerbil retina transduced with ShH10 expressing GFP. FIG. 11B depicts a fluorescent fundus image of a gerbil retina transduced with ShH10/7m8(457) expressing GFP. FIGS. 11C-11E depict single-channel immunofluorescence images of transverse sections of gerbil retinas transduced with ShH10 (top panel) or ShH10/7m8(457) (bottom panel), the cells are stained for DAPI (to detect cell nuclei) (FIG. 11C), Rhodopsin (to detect rod cells) (FIG. 11D), and GFP (FIG. 11E).

FIGS. 12A-12D shows OCT images obtained using the Heidelberg Spectralis of African Green Monkey (AGM) retina twelve weeks following intravitreal administration of 2×10¹² vg/eye of ShH10 (FIG. 12A-12B) or ShH10/7m8(457) (FIGS. 12C-12D) expressing GFP.

FIG. 13 provides a live fluorescence image of a flat-mounted AGM retina extracted twelve weeks following intravitreal administration of 2×10¹² vg/eye of ShH10/7m8(457) expressing GFP. Robust expression can be seen in the fovea (arrow) as well as in the periphery of the retina.

FIG. 14A-E provide a DIC image (FIG. 14A) as well as single-channel immunofluorescent images of a transverse section taken at the fovea of an AGM retina twelve weeks following intravitreal administration of 2×10¹² vg/eye of ShH10/7m8(457) expressing GFP. Single-channel images show staining for DAPI (to detect cell nuclei) (FIG. 14B), calbindin (to detect bipolar cells) (FIG. 14C), s-opsin (to detect s-cones) (FIG. 14D), and GFP (FIG. 14E).

FIG. 15A-15E provide a DIC image (FIG. 15A) as well as single-channel immunofluorescent images of a transverse section, taken at the periphery of an AGM retina twelve weeks following intravitreal administration of 2×10¹² vg/eye of ShH10/7m8(457) expressing GFP. Single-channel images show staining for DAPI (to detect cell nuclei) (FIG. 15B), PNA (to detect cone cells) (FIG. 15C), vimentin (to detect Muller cells) (FIG. 15D), and GFP (FIG. 15E).

FIGS. 16A-16D show the results of live imaging of luciferase in mice transduced with 1×10¹¹ vg/mouse of the ShH10 virus expressing luciferase driven by CAG promoter two weeks, four weeks and six weeks following intravenous administration of the virus. FIGS. 16A, 16B, and 16C show mouse staining at two, four, and 6 weeks, respectively, and FIG. 16D is a graph plotting the RLUs (reflecting light units) of each individual mouse numbered as 16, 17, 18, 19, 20, 21, and vehicle control at the different time points.

FIGS. 17A-17D show the results of staining for luciferase in mice transduced with 1×10¹¹ vg/mouse of the ShH10/7m8(458) virus expressing luciferase driven by CAG promoter two weeks, four weeks and six weeks following intravenous administration of the virus. FIGS. 17A, 17B, and 17C show live imaging of the mice at two weeks, four weeks and six weeks, respectively, and FIG. 17D is a graph showing the RLUs of each individual mouse numbered from 28-33 and vehicle control.

FIGS. 18A-18C provide graphs showing total luciferase expression (combined dorsal and ventral expression) (FIG. 18A), or expression from the dorsal (FIG. 18B) and ventral surface of mice at two weeks, four weeks, and six weeks following transduction with 1×10¹¹ vg/mouse of ShH10 or ShH10/7m8(458) virus expressing luciferase, or with vehicle control.

FIG. 19 is a graph showing total luciferase expression based on IVIS (in vivo imaging system) at six weeks following transduction of the indicated virus expressing luciferase.

FIGS. 20A-20C show mRNA and protein levels of luciferase and control GAPDH in liver tissue samples obtained from animals transduced by injection with the indicated virus expressing luciferase. In these figures, “ShH10/7m8” refers to ShH10/7m8(458). FIG. 20A shows a representative gel containing PCR products obtained after mRNA isolation from liver tissue samples, conversion of mRNA to cDNA, followed by PCR amplification using primers specific to either luciferase or GAPDH, followed by gel electrophoresis; FIG. 20B shows the fold change over vehicle as determined by reverse transcriptase quantitative PCR (RT-qPCR) analyses; and FIG. 20C shows the luciferase signal detected in protein extracted from liver tissue samples.

FIGS. 21A and 21B show mRNA levels of luciferase and control GAPDH in heart tissue samples obtained from animals transduced by injection with the indicated virus expressing luciferase. In these figures, “ShH10/7m8” refers to ShH10/7m8(458). FIG. 21A shows a representative gel containing PCR products obtained after mRNA isolation from heart tissue samples, conversion of mRNA to cDNA, followed by PCR amplification using primers specific to either luciferase or GAPDH, followed by gel electrophoresis; and FIG. 21B shows the fold change over vehicle of luciferase mRNA as determined by RT-qPCR analysis.

FIGS. 22A and 22B show mRNA levels of luciferase and control GAPDH in brain tissue samples obtained from animals transduced by injection with the indicated virus expressing luciferase. In these figures, “ShH10/7m8” refers to ShH10/7m8(458). FIG. 22A shows a representative gel containing PCR products obtained after mRNA isolation from heart tissue samples, conversion of mRNA to cDNA, followed by PCR amplification using primers specific to either luciferase or GAPDH, followed by gel electrophoresis; and FIG. 22B shows the fold change over vehicle of luciferase mRNA as determined by RT-qPCR analysis.

### DETAILED DESCRIPTION OF THE INVENTION

The present disclosure provides modified capsid proteins and virions and viral vectors having one or more modified or altered capsid protein, where in various embodiments, the virions exhibit: 1) increased infectivity of a retinal cell or a liver cell; 2) altered tropism; 3) increased tissue specificity for a retinal cell or a liver cell as compared to one or more other cells or tissues; 3) increased binding to heparan or heparan sulfate proteoglycans and/or the inner limiting membrane (ILM); 4) reduced and/or 5) an increased ability to infect and/or deliver a therapeutic gene product across the ILM when administered intravitreally, as compared to a corresponding virion comprising its native or wild-type capsid protein instead of a modified capsid protein disclosed herein. Also provided are pharmaceutical compositions and methods for the use of any of the compositions disclosed herein for promoting the expression of a gene in cells, e.g., retinal cells, in an individual, e.g., for the treatment or prophylaxis of a disease or disorder. These and other objects, advantages, and features of the invention will become apparent to those persons skilled in the art upon reading the details of the compositions and methods as more fully described below.

### Definitions

A “vector” as used herein refers to a macromolecule or association of macromolecules that comprises or associates with a polynucleotide and which can be used to mediate delivery of the polynucleotide to a cell. Illustrative vectors include, for example, plasmids, viral vectors, liposomes, and other gene delivery vehicles.

The term “AAV” is an abbreviation for adeno-associated virus, and may be used to refer to the virus itself or derivatives thereof. The term covers all subtypes and both naturally occurring and recombinant forms, except where required otherwise. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC 002077 (AAV-1), AF063497 (AAV-1), NC_001401 (AAV-2), AF043303 (AAV-2), NC_001729 (AAV-3), NC_-001829 (AAV-4), U89790 (AAV-4), NC_006152 (AAV-5), AF028704 and AAB95450 (AAV-6), AF513851 (AAV-7), AF513852 (AAV-8), and NC_006261 (AAV-8), the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99:11854; Moris et al. (2004) Virology 33:375-383; international patent publications WO 00/28061, WO 99/61601, WO 98/11244; and U.S. Pat. No. 6,156,303. In addition, polynucleotide sequences encoding any of the capsid proteins may be readily generated based on the amino acid sequence and the known genetic code, including codon-optimized sequences.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome such as a transgene to be delivered to a mammalian cell), it is typically referred to as a “rAAV vector particle” or simply a “rAAV vector”. Thus, production of rAAV particle necessarily includes production of rAAV vector, as such a vector is contained within a rAAV particle.

The term “replication defective” as used herein relative to an AAV viral vector of the invention means the AAV vector cannot independently replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate further.

An “AAV variant” or “AAV mutant” as used herein refers to a viral particle composed of: a) a variant AAV capsid protein, where the variant AAV capsid protein comprises at least one amino acid difference (e.g., amino acid substitution, amino acid insertion, amino acid deletion) relative to a corresponding parental AAV capsid protein, where the AAV capsid protein does not correspond to the amino acid sequence present of a naturally occurring AAV capsid protein; and, optionally, b) a heterologous nucleic acid comprising a nucleotide sequence encoding a heterologous gene product, wherein the variant AAV capsid protein confers increased binding to heparan or a heparan sulfate proteoglycan as compared to the binding by an AAV virion comprising the corresponding parental AAV capsid protein. In certain embodiments, the variant capsid protein confers: a) increased infectivity of a retinal cell compared to the infectivity of the retinal cell by an AAV virion comprising the corresponding parental AAV capsid protein; b) altered cellular tropism as compared to the tropism of an AAV virion comprising the corresponding parental AAV capsid protein; and/or c) an increased ability to bind and/or cross the ILM as compared to an AAV virion comprising the corresponding parental AAV capsid protein.

The abbreviation “rAAV” refers to recombinant adeno-associated virus, also referred to as a recombinant AAV vector (or “rAAV vector”). A “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans. For example, a plasmid or other expression vector comprising nucleotide sequences encoding one or more adenoviral proteins is transfected into a producer cell along with an rAAV vector.

An “infectious” virus or viral particle is one that comprises a competently assembled viral capsid and is capable of delivering a polynucleotide component into a cell for which the viral species is tropic. The term does not necessarily imply any replication capacity of the virus. Assays for counting infectious viral particles are described elsewhere in this disclosure and in the art. Viral infectivity can be expressed as the ratio of infectious viral particles to total viral particles. Methods of determining the ratio of infectious viral particle to total viral particle are known in the art. See, e.g., Grainger et al. (2005) Mol. Ther. 11:S337 (describing a TCID50 infectious titer assay); and Zolotukhin et al. (1999) Gene Ther. 6:973. See also the Examples.

A “replication-competent” virus (e.g. a replication-competent AAV) refers to a phenotypically wild-type virus that is infectious, and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) even in the presence of helper functions, by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 10² rAAV particles, less than about 1 rcAAV per 10⁴ rAAV particles, less than about 1 rcAAV per 10⁸ rAAV particles, less than about 1 rcAAV per 10¹² rAAV particles, or no rcAAV).

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

A “gene” refers to a polynucleotide containing at least one open reading frame that is capable of encoding a particular gene product after being transcribed, and sometimes also translated. The term “gene” or “coding sequence” refers to a nucleotide sequence in vitro or in vivo that encodes a gene product. In some instances, the gene consists or consists essentially of coding sequence, that is, sequence that encodes the gene product. In other instances, the gene comprises additional, non-coding, sequence. For example, the gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

A “gene product” is a molecule resulting from expression of a particular gene. Gene products include, e.g., a polypeptide, an aptamer, an interfering RNA, an mRNA, and the like. In particular embodiments, a “gene product” is a polypeptide, peptide, protein or interfering RNA including short interfering RNA (siRNA), miRNA or small hairpin RNA (shRNA). In particular embodiments, a gene product is a therapeutic gene product, e.g., a therapeutic protein.

As used herein, a “therapeutic gene” refers to a gene that, when expressed, produces a therapeutic gene product that confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include, but are not limited to, genes that correct a genetic deficiency in a cell or mammal.

As used herein, a “transgene” is a gene that is delivered to a cell by a vector.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a gene product of interest, and is used for effecting the expression of the gene product in an intended target cell. An expression vector also comprises control elements operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

As used herein, the terms “polypeptide,” “peptide,” and “protein” refer to polymers of amino acids of any length. The terms also encompass an amino acid polymer that has been modified; for example, disulfide bond formation, glycosylation, lipidation, phosphorylation, or conjugation with a labeling component.

By “comprising” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g. poly-adenylation sequence, enhancer elements, other genes, linker domains, etc.

By “consisting essentially of”, it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g. linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g. 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of”, it is meant the exclusion from the composition, method, or kit of any element, step, or ingredient not specified in the claim. For example, an expression cassette “consisting of” a gene encoding a therapeutic polypeptide operably linked to a promoter, and a polyadenylation sequence consists only of the promoter, polynucleotide sequence encoding the therapeutic polypeptide, and polyadenlyation sequence. As another example, a polypeptide “consisting of” a recited sequence contains only the recited sequence.

An “expression vector” as used herein encompasses a vector, e.g. plasmid, minicircle, viral vector, liposome, and the like as discussed above or as known in the art, comprising a polynucleotide which encodes a gene product of interest, and is used for effecting the expression of a gene product in an intended target cell. An expression vector also comprises control elements operatively linked to the encoding region to facilitate expression of the gene product in the target. The combination of control elements, e.g. promoters, enhancers, UTRs, miRNA targeting sequences, etc., and a gene or genes to which they are operably linked for expression is sometimes referred to as an “expression cassette.” Many such control elements are known and available in the art or can be readily constructed from components that are available in the art.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

An “enhancer” as used herein encompasses a cis-acting element that stimulates or inhibits transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region.

A “termination signal sequence” as used herein encompasses any genetic element that causes RNA polymerase to terminate transcription, such as for example a polyadenylation signal sequence.

A “polyadenylation signal sequence” as used herein encompasses a recognition region necessary for endonuclease cleavage of an RNA transcript that is followed by the polyadenylation consensus sequence AATAAA. A polyadenylation signal sequence provides a “polyA site”, i.e. a site on a RNA transcript to which adenine residues will be added by post-transcriptional polyadenylation.

As used herein, the terms “operatively linked” or “operably linked” refers to a juxtaposition of genetic elements, e.g. promoter, enhancer, termination signal sequence, polyadenylation sequence, etc., wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained. As used herein, the term “heterologous” means derived from a genotypically distinct entity from that of the rest of the entity to which it is being compared. For example, a polynucleotide introduced by genetic engineering techniques into a plasmid or vector derived from a different species is a heterologous polynucleotide. As another example, a promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

The term “endogenous” as used herein with reference to a nucleotide molecule or gene product refers to a nucleic acid sequence, e.g. gene or genetic element, or gene product, e.g. RNA, protein, that is naturally occurring in or associated with a host virus or cell.

The term “native” as used herein refers to a nucleotide sequence, e.g. gene, or gene product, e.g. RNA, protein, that is present in a wildtype virus or cell. The term “variant” as used herein refers to a mutant of a reference polynucleotide or polypeptide sequence, for example a native polynucleotide or polypeptide sequence, i.e. having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g. a native polynucleotide or polypeptide sequence. For example, a variant may be a polynucleotide having a sequence identity of 70% or more with a full length native polynucleotide sequence, e.g. an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g. an identity of 75% or 80% or more, such as 85%, 90%, or 95% or more, for example, 98% or 99% identity with the full length native polypeptide sequence. Variants may also include variant fragments of a reference, e.g. native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

As used herein, the terms “biological activity” and “biologically active” refer to the activity attributed to a particular biological element in a cell. For example, the “biological activity” of an “immunoglobulin”, “antibody” or fragment or variant thereof refers to the ability to bind an antigenic determinant and thereby facilitate immunological function. As another example, the biological activity of a polypeptide or functional fragment or variant thereof refers to the ability of the polypeptide or functional fragment or variant thereof to carry out its native functions of, e.g., binding, enzymatic activity, etc. As a third example, the biological activity of a gene regulatory element, e.g. promoter, enhancer, kozak sequence, and the like, refers to the ability of the regulatory element or functional fragment or variant thereof to regulate, i.e. promote, enhance, or activate the translation of, respectively, the expression of the gene to which it is operably linked.

The terms “administering” or “introducing”, as used herein, refer to delivery of a vector for recombinant gene or protein expression to a cell, to cells and/or organs of a subject, or to a subject. Such administering or introducing may take place in vivo, in vitro or ex vivo. A vector for expression of a gene product may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus; or transduction, which typically means stable infection of a cell with a virus or the transfer of genetic material from one microorganism to another by way of a viral agent (e.g., a bacteriophage).

“Transformation” is typically used to refer to bacteria comprising heterologous DNA or cells which express an oncogene and have therefore been converted into a continuous growth mode such as tumor cells. A vector used to “transform” a cell may be a plasmid, virus or other vehicle.

Typically, a cell is referred to as “transduced”, “infected”; “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

The term “host cell”, as used herein refers to a cell which has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

The terms “treatment”, “treating” and the like are used herein to generally mean obtaining a desired pharmacologic and/or physiologic effect. The effect may be prophylactic in terms of completely or partially preventing a disease or symptom thereof, e.g. reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

The various compositions and methods of the invention are described below. Although particular compositions and methods are exemplified herein, it is understood that any of a number of alternative compositions and methods are applicable and suitable for use in practicing the invention. It will also be understood that an evaluation of the expression constructs and methods of the invention may be carried out using procedures standard in the art.

The practice of the present invention will employ, unless otherwise indicated, conventional techniques of cell biology, molecular biology (including recombinant techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing-herein is to be construed as an admission that the present invention is not entitled to antedate such publication by virtue of prior invention. Further, the dates of publication provided may be different from the actual publication dates which may need to be independently confirmed.

Unless otherwise indicated, all terms used herein have the same meaning as they would to one skilled in the art and the practice of the present invention will employ, conventional techniques of microbiology and recombinant DNA technology, which are within the knowledge of those of skill of the art.

### Variant AAV Capsid Polypeptides

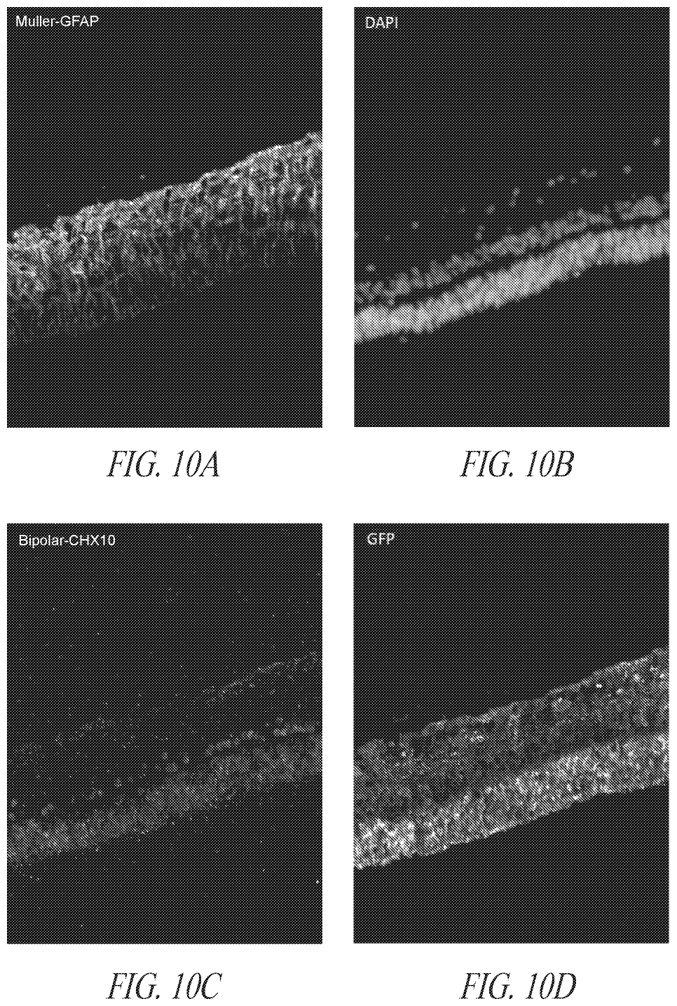

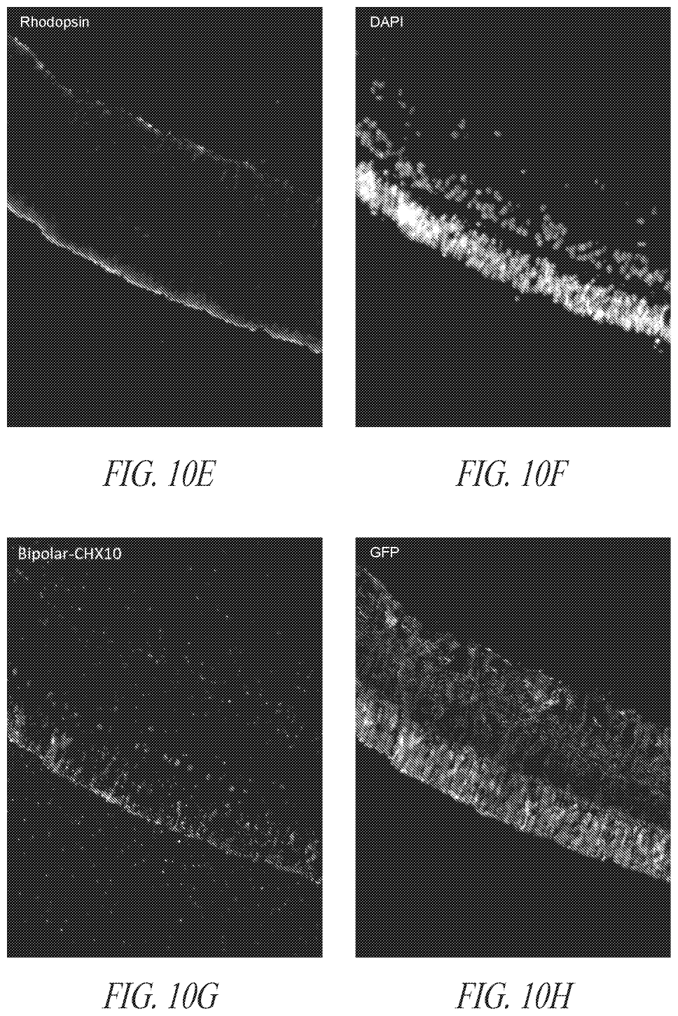

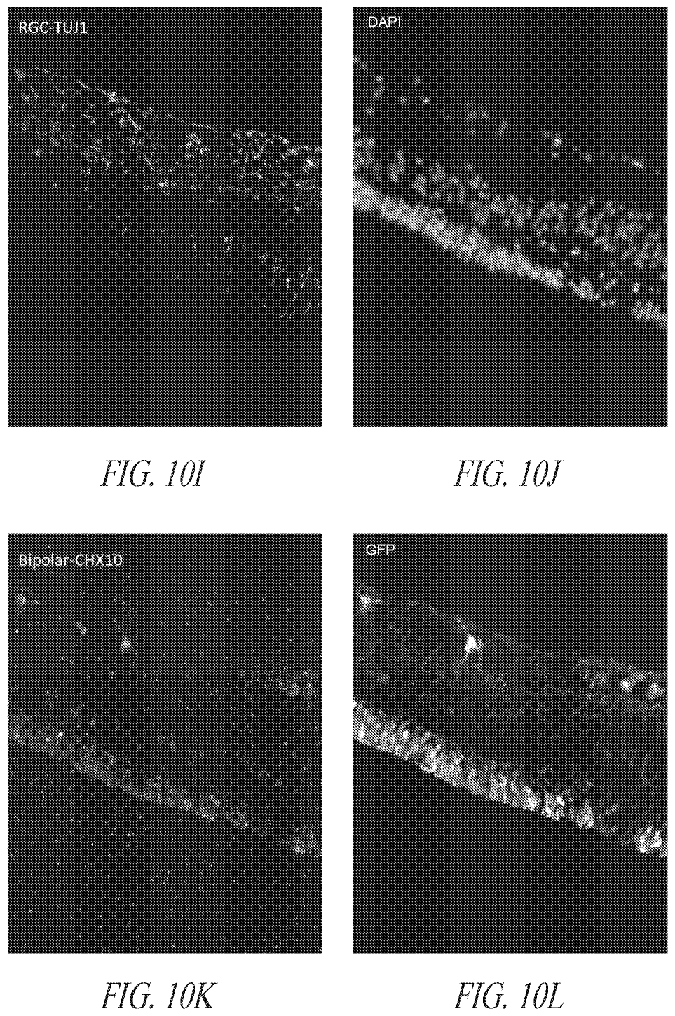

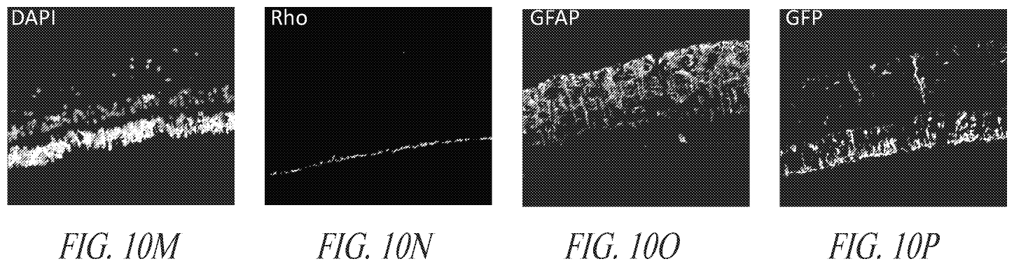

The present disclosure provides variant AAV capsid proteins, e.g., VP1 proteins, where the variant AAV capsid protein comprises one or more amino acid modifications as compared to the corresponding wild-type AAV or parental AAV. In particular embodiments, the variant AAV capsid proteins comprise an amino acid insertion between two adjacent amino acid residues of the AAV capsid protein. In particular embodiments, the variant AAV capsid protein comprises an insertion within a capsid protein, e.g., VP1, of AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV rh.10, avian AAV, bovine AAV, canine AAV, equine AAV, primate AAV, non-primate AAV, bovine AAV, AAV.7m8, AAVShH10, AAV2.5T, AAV2.5T/7m8, AAV9/7m8, and AAVS/7m8. AAV2.5T capsid proteins and virions are described in U.S. Pat. No. 9,233,131, in which the VP1-encoding amino acid sequences of AAV2.5T is provided as SEQ ID NO:42 and FIGS. 10A-B. AAV.7m8 capsid proteins are described in U.S. Pat. No. 9,193,956. AAV.7m8 includes a 7m8 insert between amino acids 587 and 588 of the wildtype AAV2 genome. AAV2.5T/7m8 capsid proteins correspond to AAV2.5T capsid proteins further comprising a 7m8 insert.

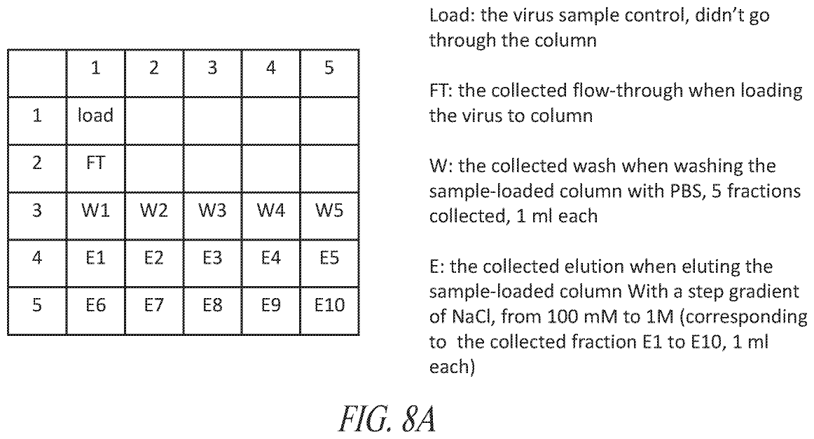

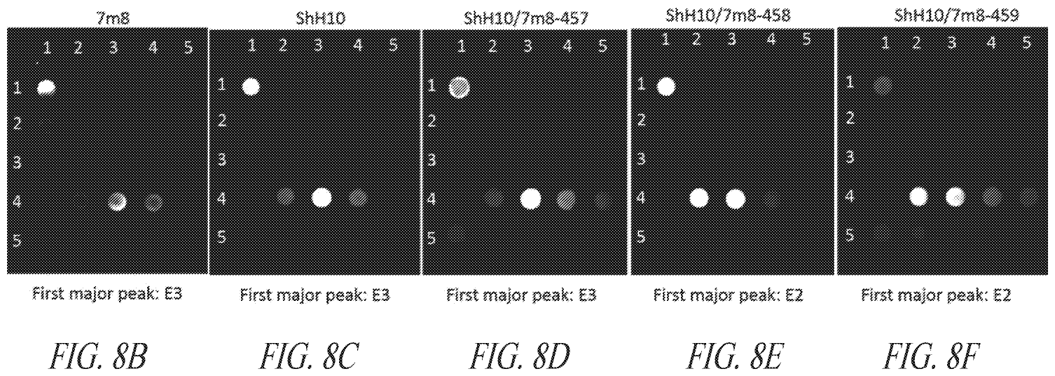

In certain embodiments, the parental AAV capsid protein is an AAVShH10, AAV1, or AAV6 capsid protein. SEQ ID NO:3 and FIGS. 8A-8C of U.S. Patent Application Publication No. 20120164106 show the amino acid sequence of the AAVShH10 capsid protein, which is also described in Klimczak, R. R. et al., PLOS One 4(10):e7467 (Oct. 14, 2009). The amino acid sequence of the AAV1 capsid protein can be found GENBANK Accession No. NP_049542 (SEQ ID NO:31). The amino acid sequence of the AAV6 capsid protein can be found GENBANK Accession No. AAB95450 (SEQ ID NO:32). While reference is made herein to amino acid modifications of capsid proteins (including specific amino acid insertions) using the amino acid numbering corresponding to the AAVShH10 VP1 capsid protein, it is understood that any of these amino acid modifications may also be introduced in the capsid proteins of AAVs of other serotypes, e.g., at positions corresponding to those of AAVShH10.

In particular embodiments, the variant capsid protein, when present in an AAV virion, confers increased infectivity of a retinal cell or a liver cell as compared to the infectivity of the retinal cell by an AAV virion comprising the corresponding parental AAV capsid protein. In some cases, the retinal cell is a photoreceptor cell (e.g., rods or cones). In other cases, the retinal cell is an RGC. In other cases, the retinal cell is an RPE cell. In other cases, the retinal cell is a Muller cell. Other retinal cells include amacrine cells, bipolar cells, and horizontal cells. In particular embodiments, the variant capsid protein, when present in an AAV virion, confers altered tropism as compared to the tropism of the retinal cell by an AAV virion comprising the corresponding parental AAV capsid protein. In particular embodiments, the variant capsid protein, when present in an AAV virion, confers increased binding to heparan or heparan sulfate, and/or increased ability to bind and cross the inner limiting membrane following intravitreal injection, as compared to an AAV virion comprising the corresponding parental AAV capsid protein.

In certain embodiments, the variant capsid protein, e.g., VP1, includes an insertion of a peptide of from about 5 amino acids to about 11 amino acids in length. In particular embodiments, the insertion peptide has a length of 5 amino acids, 6 amino acids, 7 amino acids, 8 amino acids, 9 amino acids, 10 amino acids, or 11 amino acids. These insertions are collectively referred to as “7m8 insertions.”

In certain embodiments, the 7m8 insertion peptide comprises an amino acid sequence of any one of the formulas set forth herein. For example, an insertion peptide can be a peptide of from 5 to 11 amino acids in length, where the insertion peptide is of Formula I:

Y₁Y₂X₁X₂X₃X₄X₅X₆X₇Y₃Y₄ (SEQ ID NO:1)

where:
each of Y₁-Y₄ is independently absent or present and, if present, is independently selected from Ala, Leu, Gly, Ser, and Thr;
X₁ is absent or present and, if present, is selected from Leu, Asn, and Lys;
X₂ is selected from Gly, Glu, Ala, and Asp;
X₃ is selected from Glu, Thr, Gly, and Pro;
X₄ is selected from Thr, Ile, Gln, and Lys;
X₅ is selected from Thr and Ala;
X₆ is selected from Arg, Asn, and Thr; and
X₇ is absent or present and, if present, is selected from Pro and Asn.

As another example, a 7m8 insertion peptide can be a peptide of from 5 to 11 amino acids in length, where the insertion peptide is of Formula IIa:

Y₁Y₂X₁X₂X₃X₄X₅X₆X₇Y₃Y₄ (SEQ ID NO:2)

where:
each of Y₁-Y₄ are independently absent or present and, if present, is independently selected from Ala, Leu, Gly, Ser, and Thr;
each of X₁-X₄ is any amino acid;
X₅ is Thr;
X₆ is Arg; and
and X₇ is Pro.

As another example, an insertion peptide can be a peptide of from 5 to 11 amino acids in length, where the insertion peptide is of Formula IIb:

Y₁Y₂X₁X₂X₃X₄X₅X₆X₇Y₃Y₄ (SEQ ID NO:3)

where:
each of Y₁-Y₄ is independently absent or present and, if present, is independently selected from Ala, Leu, Gly, Ser, and Thr;
X₁ is absent or present and, if present, is selected from Leu and Asn;
X₂ is absent or present and, if present, is selected from Gly and Glu;
X₃ is selected from Glu and Thr;
X₄ is selected from Thr and Ile;
X₅ is Thr;
X₆ is Arg; and
X₇ is Pro.

As another example, an 7m8 insertion peptide can be a peptide of from 5 to 11 amino acids in length, where the insertion peptide is of Formula III:

Y₁Y₂X₁X₂X₃X₄X₅X₆X₇Y₃Y₄ (SEQ ID NO:4)

where:
each of Y₁-Y₄ is independent absent or present and, if present, is independently selected from Ala, Leu, Gly, Ser, and Thr;
X₁ is absent or present and, if present, is Lys;
X₂ is selected from Ala and Asp;
X₃ is selected from Gly and Pro;
X₄ is selected from Gln and Lys;
X₅ is selected from Thr and Ala;
X₆ is selected from Asn and Thr; and
X₇ is absent or present and, if present, is Asn.

As another example, an insertion peptide can be a peptide of from 5 to 11 amino acids in length, where the insertion peptide is of Formula IV:

Y₁Y₂X₁X₂X₃X₄X₅X₆X₇Y₃Y₄ (SEQ ID NO:5)

where:
each of Y₁-Y₄ is independently absent or present and, if present, is independently selected from Ala, Leu, Gly, Ser, and Thr;
X₁ is absent or present, if present, is a positively charged amino acid or an uncharged amino acid; or is selected from Leu, Asn, Arg, Ala, Ser, and Lys;
X₂ is a negatively charged amino acid or an uncharged amino acid; or is selected from Gly, Glu, Ala, Val, Thr, and Asp;
X₃ is a negatively charged amino acid or an uncharged amino acid; or is selected from Glu, Thr, Gly, Asp, or Pro;
X₄ is selected from Thr, Ile, Gly, Lys, Asp, and Gln;
X₅ is a polar amino acid, an alcohol (an amino acid having a free hydroxyl group), or a hydrophobic amino acid; or is selected from Thr, Ser, Val, and Ala;
X₆ is a positively charged amino acid or an uncharged amino acid; or is selected from Arg, Val, Lys, Pro, Thr, and Asn; and
X₇ is absent or present and, if present, is a positively charged amino acid or an uncharged amino acid; or is selected from Pro, Gly, Phe, Asn, and Arg.

As non-limiting examples, the 7m8 insertion peptide can comprise or consist of an amino acid sequence selected from LGETTRP (SEQ ID NO:6), NETITRP (SEQ ID NO:7), KAGQANN (SEQ ID NO:8), KDPKTTN (SEQ ID NO:9), KDTDTTR (SEQ ID NO:10), RAGGSVG (SEQ ID NO:11), AVDTTKF (SEQ ID NO:12), and STGKVPN (SEQ ID NO:13).

In some cases, the 7m8 insertion peptide has from 1 to 4 spacer amino acids (Y₁-Y₄) at the amino terminus and/or at the carboxyl terminus of any one of LGETTRP (SEQ ID NO:6), NETITRP (SEQ ID NO:7), KAGQANN (SEQ ID NO:8), KDPKTTN (SEQ ID NO:9), KDTDTTR (SEQ ID NO:10), RAGGSVG (SEQ ID NO:11), AVDTTKF (SEQ ID NO:12), and STGKVPN (SEQ ID NO:13). Suitable spacer amino acids include, but are not limited to, leucine, alanine, glycine, and serine.

For example, in some cases, a 7m8 insertion peptide has one of the following amino acid sequences: LALGETTRPA (SEQ ID NO:14); LANETITRPA (SEQ ID NO:15), LAKAGQANNA (SEQ ID NO:16), LAKDPKTTNA (SEQ ID NO:17), LAKDTDTTRA (SEQ ID NO:18), LARAGGSVGA (SEQ ID NO:19), LAAVDTTKFA (SEQ ID NO:20), and LASTGKVPNA (SEQ ID NO:21). As another example, in some cases, a 7m8 insertion peptide has one of the following amino acid sequences: AALGETTRPA (SEQ ID NO:22); AANETITRPA (SEQ ID NO:23), AAKAGQANNA (SEQ ID NO:24), and AAKDPKTTNA (SEQ ID NO:25). As yet another example, in some cases, a 7m8 insertion peptide has one of the following amino acid sequences: GLGETTRPA (SEQ ID NO:26); GNETITRPA (SEQ ID NO:27), GKAGQANNA (SEQ ID NO:28), and GKDPKTTNA (SEQ ID NO:29). As another example, in some cases, an insertion peptide comprises one of KDTDTTR (SEQ ID NO:10), RAGGSVG (SEQ ID NO:11), AVDTTKF (SEQ ID NO:12), and STGKVPN (SEQ ID NO:13), flanked on the C-terminus by AA and on the N-terminus by A; or comprises one of KDTDTTR (SEQ ID NO:10), RAGGSVG (SEQ ID NO:11), AVDTTKF (SEQ ID NO:12), and STGKVPN (SEQ ID NO:13) flanked on the C-terminus by G and on the N-terminus by A. In certain embodiments, the 7m8 is a random sequence of five to 12 amino acid residues.

In certain embodiment, the 7m8 amino acid insert comprises or consists of the following amino acid sequence: LGETTRP (SEQ ID NO:6). In particular embodiments, the 7m8 insert comprises or consists of the amino acid sequence: LALGETTRPA (SEQ ID NO:14), or a fragment comprising at least five, at least six, at least seven, at least eight, or at least nine consecutive amino acids thereof. In particular embodiments, the 7m8 insert comprises or consists of an amino acid sequence having at least 80%, at least 85%, or at least 90% homology to the amino acid sequence: LALGETTRPA (SEQ ID NO:14), or a fragment comprising at least five, at least six, at least seven, at least eight, or at least nine consecutive amino acids thereof. In some embodiments, a capsid protein includes an m78 insertion comprising an amino acid sequence having at least about 85%, at least about 90%, at least about 95%, at least about 98%, at least about 99%, or 100%, amino acid sequence identity to an amino acid sequence selected from LGETTRP (SEQ ID NO:6) and LALGETTRPA (SEQ ID NO:14). In particular embodiments, any of these inserts are present within amino acid residues 450-464 of AAVShH10, AAV1, or AAV6, e.g., inserted immediately C-terminal to amino acid residue 450, 451, 452, 453, 454, 455, 456, 457, 458, 459, 460, 461, 462, 463 or 464.

In some embodiments, a subject variant AAV capsid does not include any other amino acid substitutions, insertions, or deletions, other than the 7m8 insertion relative to a corresponding parental AAV capsid protein. In other embodiments, a subject variant AAV capsid includes from 1 to about 25 amino acid insertions, deletions, or substitutions, compared to the parental AAV capsid protein, in addition to the 7m8 insertion relative to a corresponding parental AAV capsid protein. In some embodiments, a subject variant capsid polypeptide does not include one, two, three, or four, of the following amino acid substitutions: Y273F, Y444F, Y500F, and Y730F. In some embodiments, a subject variant capsid polypeptide comprises, in addition to a 7m8 insertion peptide, one, two, three, or four, of the following amino acid substitutions: Y273F, Y444F, Y500F, and Y730F.

In some embodiments, a variant AAV capsid polypeptide is a chimeric capsid, e.g., the capsid comprises a portion of an AAV capsid of a first AAV serotype and a portion of an AAV capsid of a second serotype; and comprises a 7m8 insertion relative to a corresponding parental AAV capsid protein.

In some embodiments, a subject variant capsid protein comprises an amino acid sequence having at least about 85%, at least about 90%, at least about 95%, at least about 98%, or at least about 99%, amino acid sequence identity to a wild type or parental capsid protein, e.g., VP1; and a 7m8 insertion relative to the corresponding wild type or parental AAV capsid protein.

In some embodiments, a subject variant capsid protein is isolated, e.g., purified. In some cases, a subject variant capsid protein is included in an AAV vector, which is also provided. As described in detail below, a subject variant capsid protein can be included in a recombinant AAV virion.

The 7m8 insert may be inserted at various sites within the AAV capsid protein. In particular embodiments, the variant capsid protein is an AAVShH10 capsid protein, e.g., VP1, and the 7m8 insertion site is in the variable region IV of the AAVShH10 capsid protein. AAVShH10, derived from an AAV6 parent serotype from the shuffled (ShH) library, has been shown to have increased specificity and efficiency for transducing Muller cells as compared to wild type AAV6 or AAV2. (Klimczak, R. R. et al., PLOS One 4(10):e7467 (Oct. 14, 2009)). The AAVShH10 capsid is alternatively referred to herein as “ShH10”; the terms are used interchangeably. The wild-type AAV1 virus is closely related to AAV6, with an identical capsid amino acid sequence, but for 5 amino acid substitutions. The amino acid sequence of AAV1 VP1 protein may be found GENBANK Accession No. NP_049542 (SEQ ID NO:31), and the amino acid sequence of the AAV6 VP1 protein may be found at GENBANK Accession No. AAB95450 (SEQ ID NO:32). In certain other embodiments, the variant capsid is AAV1, AAV6 or any other AAV serotype or derivate, including but not limited to those described herein, and the 7m8 insertion if present in a region of the AAV that corresponds to the variable region IV of AAVShH10. In certain embodiments, the 7m8 insertion is present at or near the tip of the 3-fold protusion, at or near the basic amino acid residue 459, or at or near the heparan sulfate proteoglycan binding site of AAVShH10 capsid protein, or a corresponding site of another AAV serotype or derivative. In particular embodiments, “near” indicates within two, within three, within four, or within five amino acid residues of the indicated amino acid or site. For example, the 7m8 insertion site can be within amino acids 450-460 or amino acids 450-464 of AAVShH10 capsid protein, e.g., between two adjacent amino acids selected from 450 and 451, 451 and 452, 452 and 453, 453 and 454, 454 and 455, 455 and 456, 456 and 457, 457 and 458, 458 and 459, 459 and 460, 460 and 461, 461 and 462, 462 and 463, or 463 and 464 of AAVShH10 capsid protein, e.g., VP1, or the corresponding amino acid residues in any other AAV capsid protein, e.g., VP1. In particular embodiments, the insertion is between amino acid residues 457 and 458 (ShH10/7m8(457)), 458 and 459 (ShH10/7m8(458)), or 459 and 460 (ShH10/7m8(459)) of AAVShH10.

In particular embodiments, the modified capsid protein is not disclosed in U.S. Pat. Nos. 9,441,244, 9,233,131, U.S. 200160017295, U.S. Pat. No. 7,220,577, WO 2015/168666, U.S. Pat. Nos. 7,867,484, 8,802,080, U.S. 20150005369, U.S. Pat. No. 7,172,893, WO 2015/134643, U.S. Pat. Nos. 6,962,815, 7,749,492, U.S. 20160040137, U.S. 20090317417, U.S. 20140336245, U.S. Pat. No. 7,629,322, WO 2016/133917, WO 2015/121501, U.S. Pat. Nos. 9,409,953, 8,889,641, or U.S. 20150152142.

The present disclosure also includes polynucleotides that encode one or more variant capsids described herein. In particular embodiments, the polynucleotide is an expression vector, and the expression vector comprises a polynucleotide sequence encoding a variant capsid described herein operably linked to a promoter sequence, e.g., a promoter sequence that drives expression of the polynucleotide in a cell. In particular embodiments, the promoter sequence is a tissue-specific promoter that preferentially drives expression in one or more tissue or cell type, e.g., the retina, the liver, retinal cells or hepatocytes.

The present disclosure also includes cells comprising a polynucleotide or vector that encodes a variant capsid described herein. In particular embodiments, the polynucleotide is an expression vector, and the expression vector comprises a polynucleotide sequence encoding a variant capsid described herein operably linked to a promoter sequence, e.g., a promoter sequence that drives expression of the polynucleotide in the cell. In certain embodiments, the polynucleotide or vector further comprises a sequence that encodes a rep protein, e.g., an AAV2 rep protein. In certain embodiments, the cell is a helper cell or host cell, such as, e.g., an HEK293 cell that may be used to produce virions comprising the variant capsid protein. In preparing the subject rAAV compositions, any host cells for producing rAAV virions may be employed, including, for example, mammalian cells (e.g. 293 cells), insect cells (e.g. Sf9 cells), microorganisms and yeast. Host cells can also be packaging cells in which the AAV rep and cap genes are stably maintained in the host cell or producer cells in which the AAV vector genome is stably maintained and packaged. Exemplary packaging and producer cells are derived from Sf-9, 293, A549 or HeLa cells. AAV vectors are purified and formulated using standard techniques known in the art.

### Recombinant Virions and Viral Vectors

The present invention includes recombinant viruses or virions, e.g., gene delivery vectors or gene therapy vectors that comprise a variant capsid protein of the present disclosure.

In certain embodiments, the virus or virion is a viral vector derived from a virus, e.g., an adenovirus, an adeno-associated virus (AAV), a lentivirus, a herpes virus, an alpha virus or a retrovirus, e.g., Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) or lentivirus. While embodiments encompassing the use of adeno-associated virus are described in greater detail below, it is expected that the ordinarily skilled artisan will appreciate that similar knowledge and skill in the art can be brought to bear on non-AAV gene therapy vectors as well. See, for example, the discussion of retroviral vectors in, e.g., U.S. Pat. Nos. 7,585,676 and 8,900,858, and the discussion of adenoviral vectors in, e.g. U.S. Pat. No. 7,858,367, the full disclosures of which are incorporated herein by reference. In certain embodiments, the recombinant virus or virion is infectious. In certain embodiments, the recombinant virion or virus is replication-competent. In certain embodiments, the recombinant virus or virion is replication-incompetent. In particular embodiments, the virion is an AAVShH10, AAV1, or AAV6 comprising a modified capsid protein described herein.

In some embodiments, the recombinant virion or virus is, e.g., an AAV, further comprises a polynucleotide cassette comprising a sequence that encodes a gene product, e.g., a therapeutic gene product. In certain embodiments, the sequence that encodes the gene product is operably linked to a promoter sequence. In certain embodiments, the polynucleotide cassette is flanked on the 5′ and 3′ ends by functional AAV inverted terminal repeat (ITR) sequences. By “functional AAV ITR sequences” is meant that the ITR sequences function as intended for the rescue, replication and packaging of the AAV virion. Hence, AAV ITRs for use in the gene delivery vectors of the invention need not have a wild-type nucleotide sequence, and may be altered by the insertion, deletion or substitution of nucleotides or the AAV ITRs may be derived from any of several AAV serotypes, e.g. AAV1, AAV2, AAV3, AAV4, AAV5, AAV6, AAV7, AAV8, AAV9, AAV10. Certain AAV vectors have the wild type REP and CAP genes deleted in whole or part, but retain functional flanking ITR sequences.

In certain embodiment, recombinant viruses or virions described herein comprises a heterologous nucleic acid comprising a nucleotide sequence encoding a gene product, e.g., a therapeutic gene product. In some embodiments, the gene product is an interfering RNA. In some embodiments, the gene product is an aptamer. In some embodiments, the gene product is a polypeptide. In some embodiments, the gene product is a site-specific nuclease that provide for site-specific knock-down of gene function.

Where the gene product is an interfering RNA (RNAi), suitable RNAi include RNAi that decrease the level of an apoptotic or angiogenic factor in a cell. For example, an RNAi can be an shRNA or siRNA that reduces the level of a gene product that induces or promotes apoptosis in a cell. Genes whose gene products induce or promote apoptosis are referred to herein as “pro-apoptotic genes” and the products of those genes (mRNA; protein) are referred to as “pro-apoptotic gene products.” Pro-apoptotic gene products include, e.g., Bax, Bid, Bak, and Bad gene products. See, e.g., U.S. Pat. No. 7,846,730.

Interfering RNAs could also be against an angiogenic product, for example VEGF (e.g., Cand5; see, e.g., U.S. Patent Publication No. 2011/0143400; U.S. Patent Publication No. 2008/0188437; and Reich et al. (2003) Mol. Vis. 9:210), VEGFR1 (e.g., Sirna-027; see, e.g., Kaiser et al. (2010) Am. J. Ophthalmol. 150:33; and Shen et al. (2006) Gene Ther. 13:225), or VEGFR2 (Kou et al. (2005) Biochem. 44:15064). See also, U.S. Pat. Nos. 6,649,596, 6,399,586, 5,661,135, 5,639,872, and 5,639,736; and 7,947,659 and 7,919,473.

Where the gene product is an aptamer, exemplary aptamers of interest include an aptamer against vascular endothelial growth factor (VEGF). See, e.g., Ng et al. (2006) Nat. Rev. Drug Discovery 5:123; and Lee et al. (2005) Proc. Natl. Acad. Sci. USA 102:18902. For example, a VEGF aptamer can comprise the nucleotide sequence 5′-cgcaaucagugaaugcuuauacauccg-3′ (SEQ ID NO:30). Also suitable for use is a PDGF-specific aptamer, e.g., E10030; see, e.g., Ni and Hui (2009) Ophthalmologica 223:401; and Akiyama et al. (2006) J. Cell Physiol. 207:407).

Where the gene product is a polypeptide, in certain embodiments, the polypeptide may enhance function of a retinal cell, e.g., the function of a rod or cone photoreceptor cell, a retinal ganglion cell, a Muller cell, a bipolar cell, an amacrine cell, a horizontal cell, or a retinal pigmented epithelial cell. Exemplary polypeptides include neuroprotective polypeptides (e.g., GDNF, CNTF, NT4, NGF, and NTN); anti-angiogenic polypeptides (e.g., a soluble vascular endothelial growth factor (VEGF) receptor; a VEGF-binding antibody; a VEGF-binding antibody fragment (e.g., a single chain anti-VEGF antibody); endostatin; tumstatin; angiostatin; a soluble Flt polypeptide (Lai et al. (2005) Mol. Ther. 12:659); an Fc fusion protein comprising a soluble Flt polypeptide (see, e.g., Pechan et al. (2009) Gene Ther. 16:10); pigment epithelium-derived factor (PEDF); a soluble Tie-2 receptor; etc.); tissue inhibitor of metalloproteinases-3 (TIMP-3); a light-responsive opsin, e.g., a rhodopsin; anti-apoptotic polypeptides (e.g., Bcl-2, Bcl-X1); and the like. Suitable polypeptides include, but are not limited to, glial derived neurotrophic factor (GDNF); fibroblast growth factor 2; neurturin (NTN); ciliary neurotrophic factor (CNTF); nerve growth factor (NGF); neurotrophin-4 (NT4); brain derived neurotrophic factor (BDNF); epidermal growth factor; rhodopsin; X-linked inhibitor of apoptosis; and Sonic hedgehog, as well as functional variants and fragments of any of these, including variants having at least 80%, at least 85%, at least 90%, or at least 95% sequence identity to any of these polypeptides, and fragments comprising at least 20%, at least 30%, at least 50%, at least 60%, at least 70%, at least 80%, or at least 90% of any of these polypeptides or variants thereof.

Suitable light-responsive opsins include, e.g., a light-responsive opsin as described in U.S. Patent Publication No. 2007/0261127 (e.g., ChR2; Chop2); U.S. Patent Publication No. 2001/0086421; U.S. Patent Publication No. 2010/0015095; and Diester et al. (2011) Nat. Neurosci. 14:387.

Suitable polypeptides also include, e.g., retinoschisin, retinitis pigmentosa GTPase regulator (RGPR)-interacting protein-1 (see, e.g., GenBank Accession Nos. Q96KN7, Q9EPQ2, and Q9GLM3); peripherin-2 (Prph2) (see, e.g., GenBank Accession No. NP.sub.--000313; peripherin; a retinal pigment epithelium-specific protein (RPE65), (see, e.g., GenBank AAC39660; and Morimura et al. (1998) Proc. Natl. Acad. Sci. USA 95:3088);

CHM (choroidermia (Rab escort protein 1)), a polypeptide that, when defective or missing, causes choroideremia (see, e.g., Donnelly et al. (1994) Hum. Mol. Genet. 3:1017; and van Bokhoven et al. (1994) Hum. Mol. Genet. 3:1041); and Crumbs homolog 1 (CRB1), a polypeptide that, when defective or missing, causes Leber congenital amaurosis and retinitis pigmentosa (see, e.g., den Hollander et al. (1999) Nat. Genet. 23:217; and GenBank Accession No. CAM23328).

Suitable polypeptides also include polypeptides that, when defective or missing, lead to achromotopsia, where such polypeptides include, e.g., cone photoreceptor cGMP-gated channel subunit alpha (CNGA3) (see, e.g., GenBank Accession No. NP 001289; and Booij et al. (2011) Ophthalmology 118:160-167); cone photoreceptor cGMP-gated cation channel beta-subunit (CNGB3) (see, e.g., Kohl et al. (2005) Eur J Hum Genet. 13(3):302); guanine nucleotide binding protein (G protein), alpha transducing activity polypeptide 2 (GNAT2) (ACHM4); and ACHM5; and polypeptides that, when defective or lacking, lead to various forms of color blindness (e.g., L-opsin, M-opsin, and S-opsin). See Mancuso et al. (2009) Nature 461(7265):784-787.

In some cases, a gene product of interest is a site-specific endonuclease that provide for site-specific knock-down of gene function, e.g., where the endonuclease knocks out an allele associated with a retinal disease. For example, where a dominant allele encodes a defective copy of a gene that, when wild-type, is a retinal structural protein and/or provides for normal retinal function, a site-specific endonuclease can be targeted to the defective allele and knock out the defective allele.

In addition to knocking out a defective allele, a site-specific nuclease can also be used to stimulate homologous recombination with a donor DNA that encodes a functional copy of the protein encoded by the defective allele. Thus, e.g., a subject rAAV virion can be used to deliver both a site-specific endonuclease that knocks out a defective allele, and can be used to deliver a functional copy of the defective allele, resulting in repair of the defective allele, thereby providing for production of a functional retinal protein (e.g., functional retinoschisin, functional RPE65, functional peripherin, etc.). See, e.g., Li et al. (2011) Nature 475:217. In some embodiments, a subject rAAV virion comprises a heterologous nucleotide sequence that encodes a site-specific endonuclease; and a heterologous nucleotide sequence that encodes a functional copy of a defective allele, where the functional copy encodes a functional retinal protein. Functional retinal proteins include, e.g., retinoschisin, RPE65, retinitis pigmentosa GTPase regulator (RGPR)-interacting protein-1, peripherin, peripherin-2, and the like.

Site-specific endonucleases that are suitable for use include, e.g., zinc finger nucleases (ZFNs); and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Patent Publication No. 2011/0301073.

In some embodiments, a nucleotide sequence encoding a gene product is operably linked to a constitutive promoter. In other embodiments, a nucleotide sequence encoding a gene product of interest is operably linked to an inducible promoter. In some instances, a nucleotide sequence encoding a gene product of interest is operably linked to a tissue-specific or cell type-specific regulatory element. In certain embodiments, the promoter selected from cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter.

For example, in some instances, a nucleotide sequence encoding a gene product of interest is operably linked to a photoreceptor-specific regulatory element (e.g., a photoreceptor-specific promoter), e.g., a regulatory element that confers selective expression of the operably linked gene in a photoreceptor cell. Suitable photoreceptor-specific regulatory elements include, e.g., a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225).

For example, in some instances, a nucleotide sequence encoding a gene product of interest is operably linked to a liver or hepatocyte specific regulatory element (e.g., a liver-specific promoter), e.g., a regulatory element that confers selective expression of the operably linked gene in a liver cell, e.g., a hepatocyte. Suitable liver-specific regulatory elements include, e.g., the Apolipoprotein E/C-I hepatic control region, alone or combined with the human alpha-1-antitrypsin core promoter; one or two copies of alpha 1 microglobulin/bikunin enhancer coupled to the core promoter of human thyroxine-binding globulin (TBG); or the promoter region referred to as “ET,” and described as randomly assembled hepatocyte-specific transcription factor binding sites linked to the murine transthyretin promoter, as summarized in Kattenhorn, L. M. et al, Human Gene Therapy, 2016 Dec. 1; 27(12): 947-961. Expression may be further stabilized by the inclusion of a woodchuck hepatitis virus post-transcriptional regulatory element (WPRE).

Recombinant viral vectors (e.g., rAAV virions) comprising variant capsid proteins described herein, and optionally encapsulating polynucleotide cassettes of the present disclosure, may be produced using standard methodology. For example, in the case of rAAV virions, an AAV expression vector comprising a polynucleotide cassette may be introduced into a producer cell, followed by introduction of an AAV helper construct comprising comprising a polynucleotide sequence encoding a variant capsid protein disclosed herein, and where the helper construct includes AAV coding regions capable of being expressed in the producer cell and which complement AAV helper functions absent in the AAV vector. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions comprising variant capsid proteins described herein are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety. Further compositions and methods for packaging are described in Wang et al. (US 2002/0168342), also incorporated by reference herein in its entirety.

As disclosed in the accompanying Examples, variant capsid proteins described herein confer enhanced or altered cellular tropism to virions comprising the variant capsid proteins. Accordingly, the variant capsids may be used to enhance or alter the tropism of a virus or virion in order to enhance its tropism for a desired cell type.

In particular embodiments, the virions or viral vectors comprising a variant capsid protein described herein bind to heparan or heparan sulfate proteoglycans (HSPGs), e.g., with an affinity at least 2-fold, at least 3-fold, at least 4-fold, at least 5-fold, at least 10-fold, at least 20-fold, at least 50-fold, or at least 100-fold as compared to a corresponding virion or viral vector that does not include a variant capsid protein disclosed herein.

In particular embodiments, the virions or viral vectors comprising a variant capsid protein described herein bind to ILM, e.g., with an affinity at least 2-fold, at least 3-fold, at least 4-fold, at least 5-fold, at least 10-fold, at least 20-fold, at least 50-fold, or at least 100-fold as compared to a corresponding virion or viral vector that does not include a variant capsid protein disclosed herein.

In particular embodiments, the virions or viral vectors comprising a variant capsid protein described herein have decreased immunogenicity, e.g., less than 90%, less than 80%, less than 70%, less than 60%, less than 50%, less than 40%, less than 30%, less than 20%, or less than 10%, as compared to a corresponding virion or viral vector that does not include a variant capsid protein disclosed herein.

In particular embodiments, the virions or viral vectors comprising a variant capsid protein described herein are capable of delivering a gene product to the retina when delivered via intravitreal injection, e.g., wherein they result in the expression of at least 2-fold, at least 3-fold, at least 4-fold, at least 5-fold, at least 10-fold, at least 20-fold, at least 50-fold, or at least 100-fold gene product as compared to a corresponding virion or viral vector that does not include a variant capsid protein disclosed herein. In certain embodiments, these virions or viral vectors comprising a variant capsid protein described herein are capable of selectively transducing retinal cells at a higher level that they transduce one or more other ocular cell types.

In particular embodiments, the virions or viral vectors comprising a variant capsid protein described herein are capable of delivering a gene product to the liver when delivered via intravenous injection or infusion, e.g., wherein they result in the expression of at least 2-fold, at least 3-fold, at least 4-fold, at least 5-fold, at least 10-fold, at least 20-fold, at least 50-fold, or at least 100-fold gene product as compared to a corresponding virion or viral vector that does not include a variant capsid protein disclosed herein. In certain embodiments, these virions or viral vectors comprising a variant capsid protein described herein are capable of selectively transducing liver cells at a higher level that they transduce one or more other organs, such as the heart.

In certain embodiments, the virions or viral vectors bind heparan or heparan sulfate with a binding affinity such that they are eluted from a heparan column at a salt concentration of about 0.2 M to about 0.4 M, e.g., 0.2M, 0.3M, or 0.4M, as described in the accompanying Examples.

### Pharmaceutical Compositions

Pharmaceutical compositions comprising a virion or viral vector comprising a variant capsid protein described herein and one or more pharmaceutically acceptable diluent, carrier, or excipient are also disclosed. The subject virions or vector can be combined with pharmaceutically-acceptable carriers, diluents and reagents useful in preparing a formulation that is generally safe, non-toxic, and desirable, and includes excipients that are acceptable for primate use. Such excipients can be solid, liquid, semisolid, or, in the case of an aerosol composition, gaseous. Examples of such carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile. For instances in which ocular cells are to be contacted in vivo, the subject polynucleotide cassettes or gene delivery vectors comprising the subject polynucleotide cassette can be treated as appropriate for delivery to the eye.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid to the extent that easy syringability exists. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the vector or virion in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the virion or vector against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It may be advantageous to formulate oral, ocular or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Any concentration of viral particles suitable to effectively transducer mammalian cells can be prepared. For example, the viral particles may be formulated at a concentration of 10⁸ vector genomes per ml or more, for example, 5×10⁸ vector genomes per mL; 10⁹ vector genomes per mL; 5×10⁹ vector genomes per mL, 10¹⁰ vector genomes per mL, 5×10¹⁰ vector genomes per mL; 10¹¹ vector genomes per mL; 5×10¹¹ vector genomes per mL; 10¹² vector genomes per mL; 5×10¹² vector genomes per mL; 10¹³ vector genomes per mL; 1.5×10¹³ vector genomes per mL; 3×10¹³ vector genomes per mL; 5×10¹³ vector genomes per mL; 7.5×10¹³ vector genomes per mL; 9×10¹³ vector genomes per mL; 1×10¹⁴ vector genomes per mL, 5×10¹⁴ vector genomes per mL or more, but typically not more than 1×10¹⁵ vector genomes per mL.

The subject viral vector may be formulated into any suitable unit dosage, including, without limitation, 1×10⁸ vector genomes or more, for example, 1×10⁹, 1×10¹⁰, 1×10¹¹, 1×10¹², or 1×10¹³ vector genomes or more, in certain instances, 1×10¹⁴ vector genomes, but usually no more than 4×10¹⁵ vector genomes. In some cases, the unit dosage is at most about 5×10¹⁵ vector genomes, e.g. 1×10¹⁴ vector genomes or less, for example 1×10¹³, 1×10¹², 1×10¹¹, 1×10¹⁰, or 1×10⁹ vector genomes or less, in certain instances 1×10⁸ vector genomes or less, and typically no less than 1×10⁸ vector genomes. In some cases, the unit dosage is 1×10¹⁰ to 1×10¹¹ vector genomes. In some cases, the unit dosage is 1×10¹⁰ to 3×10¹² vector genomes. In some cases, the unit dosage is 1×10⁹ to 3×10¹³ vector genomes. In some cases, the unit dosage is 1×10⁸ to 3×10¹⁴ vector genomes.

In some cases, the unit dosage of a pharmaceutical composition may be measured using multiplicity of infection (MOI). By MOI it is meant the ratio, or multiple, of vector or viral genomes to the cells to which the nucleic acid may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹°, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹°, 1×10¹¹, 1×10¹², 1×10¹³ 1×10¹⁴ 1×10¹⁵ 1×10¹⁶ 1×10¹⁷ and 1×10¹⁸ MOI.

In some aspects, the amount of pharmaceutical composition comprises about 1×10⁸ to about 1×10¹⁵ recombinant viruses, about 1×10⁹ to about 1×10¹⁴ recombinant viruses, about 1×10¹⁰ to about 1×10¹³ recombinant viruses, or about 1×10¹¹ to about 3×10¹² recombinant viruses.

The pharmaceutical compositions can be included in a container, pack, or dispenser, e.g. syringe, e.g. a prefilled syringe, together with instructions for administration.

The pharmaceutical compositions of the invention encompass any pharmaceutically acceptable salts, esters, or salts of such esters, or any other compound which, upon administration to an animal comprising a human, is capable of providing (directly or indirectly) the biologically active metabolite or residue thereof.

The term “pharmaceutically acceptable salt” refers to physiologically and pharmaceutically acceptable salts of the compounds of the invention: i.e., salts that retain the desired biological activity of the parent compound and do not impart undesired toxicological effects thereto. A variety of pharmaceutically acceptable salts are known in the art and described, e.g., in in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002).

Pharmaceutically acceptable base addition salts are formed with metals or amines, such as alkali and alkaline earth metals or organic amines. Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

The subject polynucleotide cassette or gene delivery vector, e.g., recombinant virus (virions), can be incorporated into pharmaceutical compositions for administration to mammalian patients, particularly primates and more particularly humans. The subject polynucleotide cassette or gene delivery vector, e.g. virions can be formulated in nontoxic, inert, pharmaceutically acceptable aqueous carriers, preferably at a pH ranging from 3 to 8, more preferably ranging from 6 to 8. Such sterile compositions will comprise the vector or virion containing the nucleic acid encoding the therapeutic molecule dissolved in an aqueous buffer having an acceptable pH upon reconstitution.

In some embodiments, the pharmaceutical composition provided herein comprise a therapeutically effective amount of a vector or virion in admixture with a pharmaceutically acceptable carrier and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. In particular embodiments, the pH of the buffer may be in the range of 6.5 to 7.75, preferably 7 to 7.5, and most preferably 7.2 to 7.4. The pharmaceutical composition may be formulated for various types of deliver, including, e.g., ocular delivery, intravitreal injection, intraocular injection, retinal injection, subretinal injection, parenteral administration, intravenous injection or infusion, and injection into the liver.

### Method of Enhancing or Altering Viral Tropism

As disclosed in the accompanying Example, variant capsid proteins described herein confer enhanced or altered cellular tropism or tissue specificity to virions comprising the variant capsid proteins. For example, certain variant capsid proteins described herein are associated with increased infectivity of the retina or liver, increased expression levels of gene product in the retina or liver, or increased binding to the ILM.

The variant capsids disclosed herein may be used to enhance or alter the tropism of a virus or virion in order to enhance its tropism for a desired cell type.

In particular embodiments, the virions or viral vectors comprising a variant capsid protein described herein are used to deliver a gene product to the retina. In particular embodiments, the virion has increased tropism for retinal ganglial cells (RGC) or Muller cells.

In particular embodiments, the virions or viral vectors comprising a variant capsid protein described herein are used to deliver a gene product across the ILM.

In particular embodiments, the virions or viral vectors comprising a variant capsid protein described herein are used to deliver a gene product to the liver, e.g., by intravenous injection or infusion, or by direct injection into the liver.

In particular embodiments, the virions or viral vectors comprising a variant capsid protein described herein are used to deliver a gene product to the liver via systemic delivery, e.g., by intravenous injection.

In certain embodiment, the disclosure provides a method of altering the tropism of a virus, e.g., an AAVShH10, AAV1, or AAV6 virus, comprising introducing one or more 7m8 insertion into a capsid protein of the virus at a position described herein. In related embodiments, the method comprises a method of altering the tropism of a virus, comprising incorporating a variant capsid protein disclosed herein into the virus.

### Methods of Expressing Gene Products and Treating Diseases and Disorders

Virions and viral vectors described herein, comprising a variant capsid protein described herein, may be used in delivering a transgene to a cell, e.g., cells of an animal. For example, they may be used in research, e.g., to determine the effect that the gene has on cell viability and/or function. As another example, they may be used in medicine, e.g. to treat a disorder, for example, by delivering a therapeutic gene product to a cell or tissue. Thus, in some aspects of the invention, methods are provided for the expression of a gene in cells, the method comprising contacting cells with a composition of the present disclosure. In some embodiments, contacting occurs in vitro. In some embodiments, contacting occurs in vivo, i.e., the subject composition is administered to a subject. In particular embodiments, a viral vector is administered parenterally, e.g., intravenously, orally, or by injection. In certain embodiments, it is administered to the eye by injection, e.g., administered to the retina, sub-retina or vitreous. In certain embodiments, it is administered by retinal injection, sub-retinal injection, or intravitreal injection. In certain embodiments, it is administered parenterally, e.g., via intravenous injection or infusion. In certain embodiments, it is administered locally or directly to a tissue or organ of interest, e.g., via injection into the liver.

For instances in particular embodiments in which mammalian cells are to be contacted in vitro with a subject virion or vector vector comprising a variant capsid protein disclosed herein, the cells may be from any mammalian species, e.g. rodent (e.g. mice, rats, gerbils, squirrels), rabbit, feline, canine, goat, ovine, pig, equine, bovine, primate, human. Cells may be from established cell lines, e.g. WERI cells, 661W cells, or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present disclosure are maintained for fewer than 10 passages in vitro.

If the cells are primary cells, they may be harvested from a mammal by any convenient method, e.g. whole explant, biopsy, etc. An appropriate solution may be used for dispersion or suspension of the harvested cells. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

In certain embodiments, to promote expression of the transgene, the subject virion or gene delivery vector comprising a variant capsid protein is contacted with the cells for about 30 minutes to 24 hours or more, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, 24 hours, etc. The subject virion or gene delivery vector comprising a variant capsid protein may be provided to the subject cells one or more times, e.g. one time, twice, three times, or more than three times, and the cells allowed to incubate with the agent(s) for some amount of time following each contacting event e.g. 16-24 hours, after which time the media is replaced with fresh media and the cells are cultured further. Contacting the cells may occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

In certain embodiments, an effective amount of a virion or gene delivery vector comprising a variant capsid protein is provided to produce the expression of the transgene in cells. In particular embodiments, the effective amount may be readily determined empirically, e.g., by detecting the presence or levels of transgene gene product, by detecting an effect on the viability or function of the cells, etc. In certain embodiments, an effect amount of subject virion or gene delivery vector comprising a variant capsid protein will promote equal or greater expression of the transgene in cells than the same amount of a reference virion or viral vector known in the art, e.g., AAV2.5T or AAV7m8. In certain embodiments, expression is enhanced 2-fold or more relative to the expression from a reference, or control virion or viral vector, for example 3-fold, 4-fold, or 5-fold or more, in some instances 10-fold, 20-fold or 50-fold or more, e.g. 100-fold. In certain embodiments, the enhanced expression occurs is a particular cell type, e.g., any of the ocular cells described herein.

For instances in which cells are contacted in vivo with a subject virion or gene delivery vector comprising a variant capsid protein described herein, the subject may be any mammal, e.g. rodent (e.g. mice, rats, gerbils), rabbit, feline, canine, goat, ovine, pig, equine, bovine, or primate. The methods and compositions of the present disclosure find use in the treatment of any condition that can be addressed, at least in part, by gene therapy of cells. Thus, the compositions and methods of the present disclosure find use in the treatment of individuals in need of a cell therapy. Cells include but are not limited to blood, eye, liver, kidney, heart, muscle, stomach, intestine, pancreas, and skin.

In certain embodiments, the disclosure provides a method of providing a gene product to an eye, e.g., a retina, of a subject, comprising administering to the subject by ocular injection a pharmaceutical composition comprising a recombinant virion or vector described herein, wherein the recombinant virus comprises a variant capsid provide disclosed herein and a polynucleotide sequence that encodes the gene product. In certain embodiments, the retinal cell can be a photoreceptor, a retinal ganglion cell, a Muller cell, a bipolar cell, an amacrine cell, a horizontal cell, or a retinal pigmented epithelial cell. In some cases, the retinal cell is a photoreceptor cell, e.g., a rod or cone cell. In some embodiments, delivery in by intravitreal injection, retinal injection, or sub-retinal injection.

In certain embodiments, the disclosure provides a method of treating or preventing an ocular disease or disorder in a subject in need thereof, comprising administering to one or both of the subject's eyes, e.g., by intravitreal injection, a pharmaceutical composition comprising a recombinant virion or vector described herein, wherein the recombinant virus comprises a variant capsid disclosed herein and a polynucleotide sequence that encodes a therapeutic gene product. The therapeutic gene product may be any therapeutic gene product, including but not limited to any of those described herein.

Ocular diseases that can be treated using a subject method include, but are not limited to, acute macular neuroretinopathy; Behcet's disease; choroidal neovascularization; diabetic uveitis; histoplasmosis; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy; photocoagulation, radiation retinopathy; epiretinal membrane disorders; branch retinal vein occlusion; anterior ischemic optic neuropathy; non-retinopathy diabetic retinal dysfunction; retinoschisis; retinitis pigmentosa; glaucoma; Usher syndrome, cone-rod dystrophy; Stargardt disease (fundus flavimaculatus); inherited macular degeneration; chorioretinal degeneration; Leber congenital amaurosis; congenital stationary night blindness; choroideremia; Bardet-Biedl syndrome; macular telangiectasia; Leber's hereditary optic neuropathy; retinopathy of prematurity; and disorders of color vision, including achromatopsia, protanopia, deuteranopia, and tritanopia.

In particular embodiments, the subject has been diagnosed with or is suspected of having one or more diseases or disorders selected from the group consisting of: age-related macular degeneration (AMD), wet-AMD, dry-AMD, retinal neovascularization, choroidal neovascularization, diabetic retinopathy, proliferative diabetic retinopathy, retinal vein occlusion, central retinal vein occlusion, branched retinal vein occlusion, diabetic macular edema, diabetic retinal ischemia, ischemic retinopathy, and diabetic retinal edema. In certain embodiments, the gene product inhibits neovascularization, e.g., choroidal neovascularization (CNV), in the retina of the subject. It has been found that many cellular factors play important roles in regulation in CNV generation, among which may include but are not limited to vascular endothelial growth factor (VEGF), VEGF receptor (VEGFR), platelet-derived growth factor (PDGF), hypoxia inducible factor (HIF), angiopoietin (Ang) and other cytokines, mitogen-activated protein kinases (MAPK). In particular embodiments, the gene product inhibits one or more of these cellular factors.

In particular embodiments, the gene product is an anti-VEGF protein or VEGF antagonist, such as, but not limited to the VEGF-binding proteins or functional fragments thereof disclosed in U.S. Pat. Nos. 5,712,380, 5,861,484 and 7,071,159 and VEGF-binding fusion proteins disclosed in U.S. Pat. No. 7,635,474. An anti-VEGF protein may also include the sFLT-1 protein as described in U.S. Patent Application Publication No. 2013/0323302.

The recombinant virions and viral vectors of the present disclosure may comprise a sequence encoding an anti-VEGF protein or VEGF antagonist, which refers to an agent that reduces, or inhibits, either partially or fully, the activity or production of a VEGF. A VEGF antagonist can directly or indirectly reduce or inhibit the activity or production of a specific VEGF such as VEGF₁₆₅. Furthermore, VEGF antagonists consistent with the above definition of “antagonist,” include agents that act on either a VEGF ligand or its cognate receptor so as to reduce or inhibit a VEGF-associated receptor signal. Examples of VEGF antagonists include antisense molecules, ribozymes or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors.

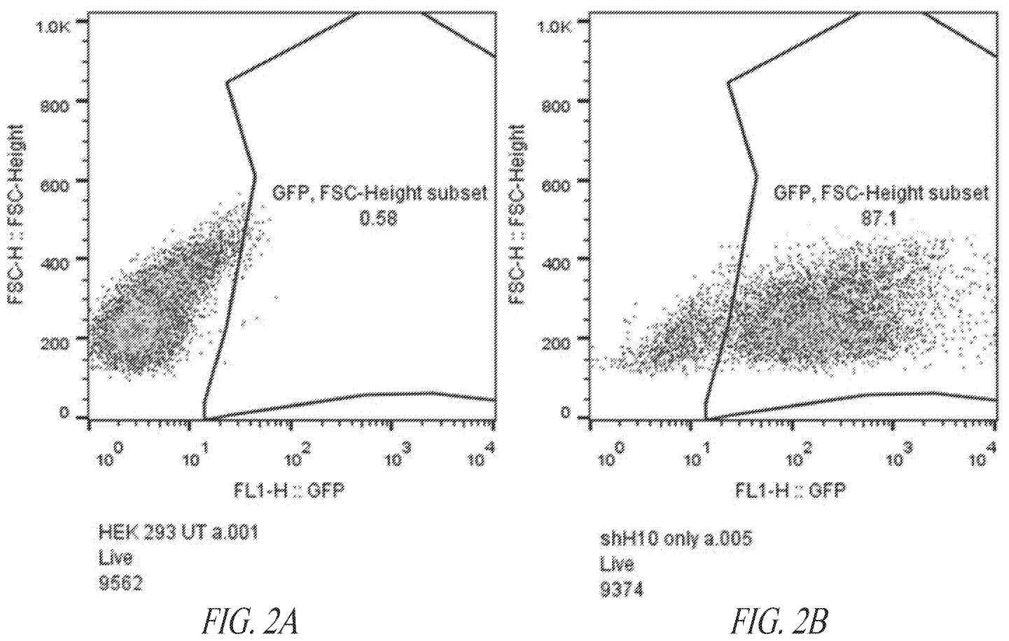

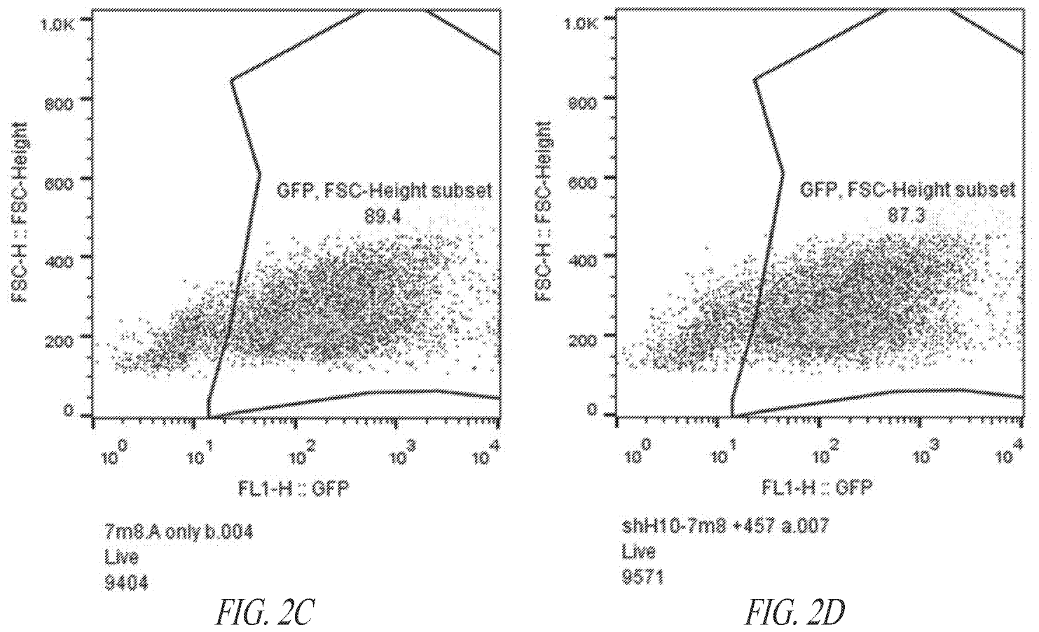

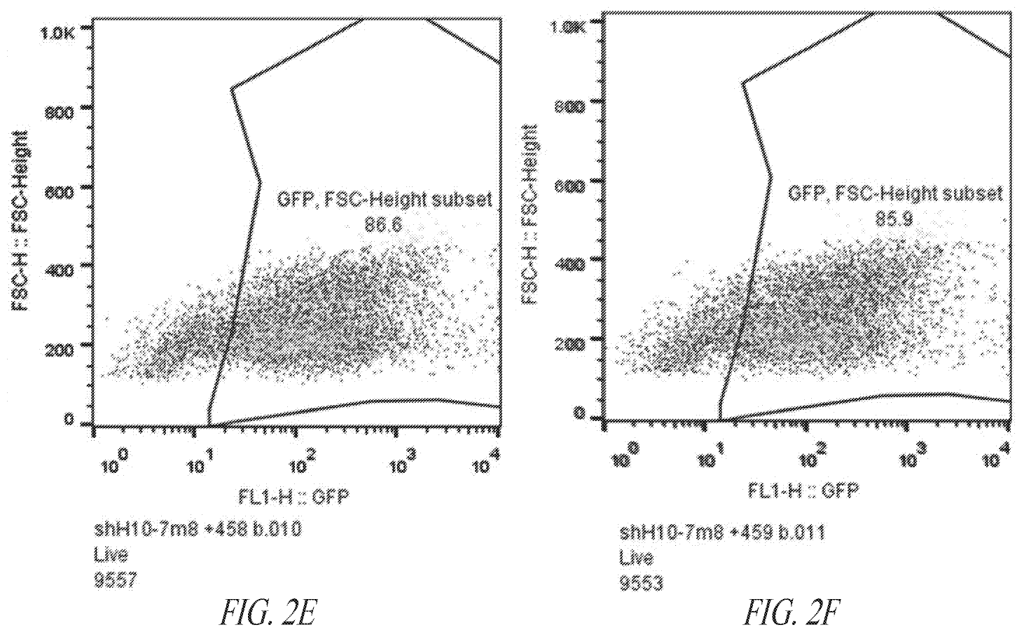

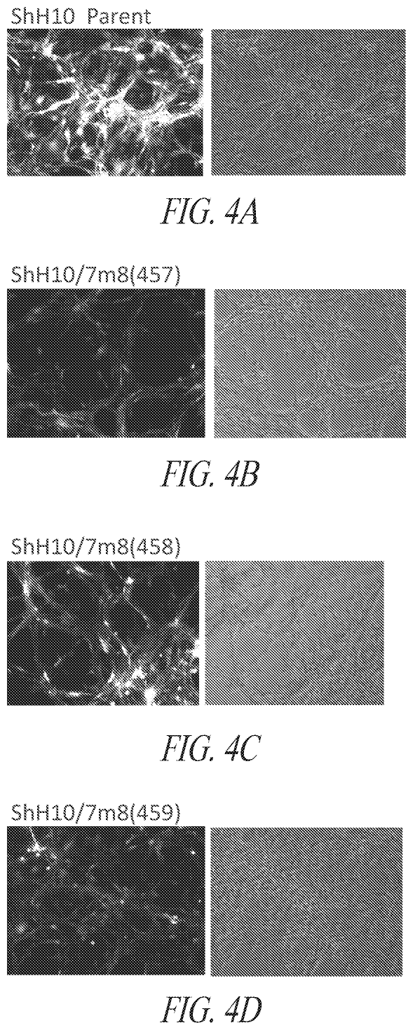

The term “VEGF” refers to a vascular endothelial growth factor that induces angiogenesis or an angiogenic process. As used herein, the term “VEGF” includes the various subtypes of VEGF (also known as vascular permeability factor (VPF) and VEGF-A) (see FIGS. 2(A) and (B) of US Patent Application Publication No. 20120100136) that arise by, e.g., alternative splicing of the VEGF-A/VPF gene including VEGF₁₂₁, VEGF₁₆₅ and VEGF₁₈₉. Further, as used herein, the term “VEGF” includes VEGF-related angiogenic factors such as PIGF (placenta growth factor), VEGF-B, VEGF-C, VEGF-D and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-1 (Flt-1) (see FIGS. 4(A) and (B) of US Patent Application Publication No. 20120100136), VEGFR-2 (KDR/Flk-1) (see FIGS. 4(C) and (D) of US Patent Application Publication No. 20120100136), or VEGFR-3 (FLT-4). The term “VEGF” can be used to refer to a “VEGF” polypeptide or a “VEGF” encoding gene or nucleic acid.

In one embodiment, the VEGF antagonist is a VEGF-A antagonist.

In one embodiment, the VEGF antagonist is ranibizumab, bevacizumab, aflibercept, KH902 VEGF receptor-Fc fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-hydroxyisovalerylshikonin, or ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLT02, Peptide B3, TG100801, sorafenib, sunitnab, G6-31 antibody, or a pharmaceutically acceptable salt thereof.

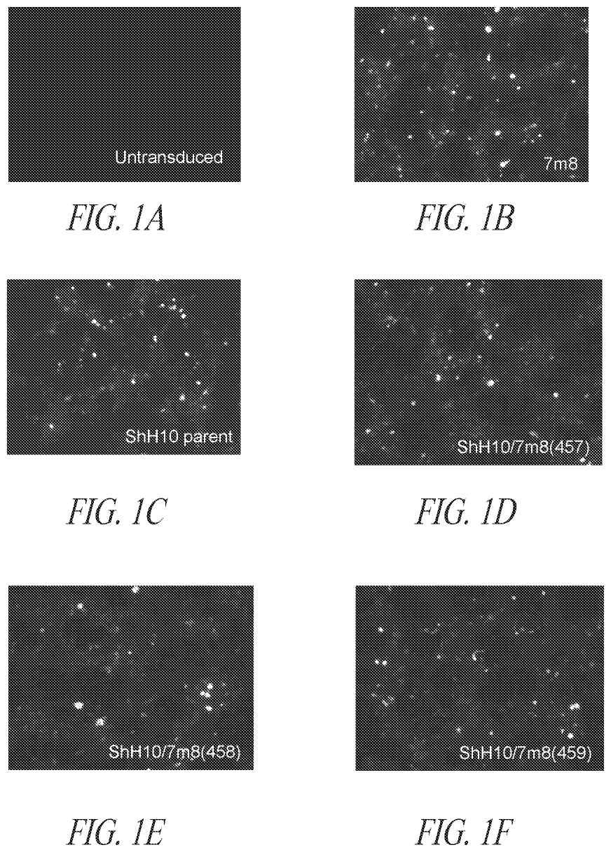

In one embodiment, the VEGF antagonist is the antibody ranibizumab or a pharmaceutically acceptable salt thereof (see U.S. Pat. No. 7,060,269 (FIG. 1) for the heavy chain and light chain variable region sequences, which is hereby incorporated by reference in its entirety). Ranibizumab is commercially available under the trademark Lucentis® (Genentech USA, Inc., a member of the Roche Group).

In another embodiment, the VEGF antagonist is the antibody bevacizumab or a pharmaceutically acceptable salt thereof (see U.S. Pat. No. 6,054,297 (FIG. 1) for the heavy chain and light chain variable region sequences, which is hereby incorporated by reference in its entirety). Bevacizumab is commercially available under the trademark Avastin® (Genentech USA, Inc., a member of the Roche Group).

In another embodiment, the VEGF antagonist is aflibercept or a pharmaceutically acceptable salt thereof (Do et al. (2009) Br J Ophthalmol. 93:144-9, which is hereby incorporated by reference in its entirety). Aflibercept is commercially available under the trademark Eyelea® (Regeneron Pharmaceuticals, Inc.).

In another embodiment, the VEGF antagonist is the naturally occurring protein sFlt-1, as described in U.S. Pat. No. 5,861,484 and that sequence described by SEQ ID NO: 109. It also includes, but is not limited to functional fragments thereof, including sequences of sFlt-1 domain 2 or those set forth in SEQ ID NO: 121 of U.S. Patent Application Publication No. 2013/0323302, as well as related constructs, such as the VEGF-binding fusion proteins disclosed in U.S. Pat. No. 7,635,474. An anti-VEGF protein may also include any of the sFLT-1 proteins, variants or fragments thereof described in U.S. Patent Application Publication No. 2013/0323302. More specifically, the VEGF binding domain (domain 2), or alternatively domain 2 of sFLT-1 plus domain 3 from sFLT1, KDR, or another family member, can be used to bind and inactivate VEGF. Such functional fragments are described in Wiesmann et al., 1997; Cell, 91: 695-704, which is incorporated herein by reference in its entirety. The terms “sFLT-1” and “a functional fragment of sFLT-1” are equivalent and used here interchangeably. “sFlt-1 protein” herein refers to a polypeptide sequence, or functional fragment thereof, with at least 90%, or more, homology to the naturally occurring human sFLT-1 sequence, such that the sFlt-1 protein or polypeptide binds to VEGF and/or the VEGF receptor.

These sequences can be expressed from DNA encoding such sequences using the genetic code, a standard technique that is understood by those skilled in the art. As can be appreciated by those with skill in the art, due to the degeneracy of the genetic code, anti-VEGF protein sequences can be readily expressed from a number of different DNA sequences.

VEGF antagonists further include nucleic acids or polypeptides having homology to any of the VEGF antagonists described herein and functional fragments of any of the VEGF antagonists or homologs. Homology refers to the % conservation of residues of an alignment between two sequences including, but not limited to functional fragments, sequences comprising insertions, deletions, substitutions, pseudofragments, pseudogenes, splice variants or artificially optimized sequences. In some cases, the VEGF antagonist may be at least about 90%, 91%, 92%, 93%, 94%, 95%, 96%, 97%, 98%, 99%, 99.9%, 99.99% or 100% homologous to the naturally occurring or parent VEGF antagonist. In some cases, the VEGF antagonist may be at most about 90%, 91%, 92%, 93%, 94%, 95%, 96%, 97%, 98%, 99%, 99.9%, 99.99% or 100% homologous to the naturally occurring or parent sequence.

In certain embodiments, virions and viral vectors comprising a variant capsid protein that confers altered tropism to the virion or viral vector is used to treat a disease or disorder of the cells for which tropism or the virion or viral vector is increased, e.g., retinal ganglial cells (RGC), Mueller cells, or to deliver a therapeutic gene product to Mueller cells. In certain embodiments, the virion or viral vector is used to treat a disease or disorder of a retinal cell selected from the group consisting of: a photoreceptor, a retinal ganglion cell, a Muller cell, a bipolar cell, an amacrine cell, a horizontal cell, or a retinal pigmented epithelial cell. In some cases, the retinal cell is a photoreceptor cell, e.g., a rod or cone cell.

In certain embodiments, the disclosure provides a method of providing a gene product to the liver of a subject, comprising parenteral administration to the subject, e.g., by intravenous injection, a pharmaceutical composition comprising a recombinant virion or vector described herein, wherein the recombinant virus comprises a variant capsid disclosed herein and a polynucleotide sequence that encodes the gene product.

In certain embodiments, the disclosure provides a method of treating or preventing a liver disease or disorder in a subject in need thereof, comprising administering to the subject, e.g., parenterally or intravenously, a pharmaceutical composition comprising a recombinant virion or vector described herein, wherein the recombinant virus comprises a variant capsid disclosed herein and a polynucleotide sequence that encodes a therapeutic gene product.

In particular embodiments, the subject has been diagnosed with or is suspected of having one or more diseases or disorders selected from the group consisting of: inherited metabolic defects, chronic viral hepatitis, liver cirrhosis, primary and metastatic liver cancer, alpha-1 antitrypsin deficiency, hemophilia B, hemophilia A, hereditary angioedema, or β-thalassemia. Gene transfer to the liver can also be used to convert this organ into a factory of secreted proteins needed to treat conditions that do not affect the liver itself

In some aspects, the disclosure provides for administering the pharmaceutical composition comprising the viral vector or virion described herein at a frequency of at least once per 3, 6, 9, 12, 18, 24 or 36 months in a human subject in need of treatment. In some aspects, the disclosure provides for administering the pharmaceutical composition comprising the viral vector or virion described herein at a frequency of at most once per 3, 6, 9, 12, 18, 24 or 36 months in a human subject in need of treatment. In some aspects, the disclosure provides for administering the pharmaceutical composition comprising the viral vector or virion described herein at a frequency less than 3 times, less than twice a year, less than once a year, less than once every two years, or less than once every three years in the human subject.

The present invention also provides methods for treating or preventing a disease or disorder, e.g., an ocular disease or disorder, or a liver disease or disorder, that include administering to a subject in need thereof a viral vector or virion comprising a modified capsid and encoding a therapeutic gene product as described herein, in combination with one or more additional therapeutic agents.

In certain embodiments, the viral vector of the present invention is administered concurrently or during an overlapping time period with the additional therapeutic agent, while in other embodiments, either the viral vector is administered first or the additional therapeutic agent is delivered first, a time period is allowed to pass, and then the other of the viral vector or the additional agent is administered. In particular embodiments, the time period is at least one day, at least one week, at least two weeks, at least one month, at least two months, at least four months, at least six months, at least one year, at least eighteen months, at least two years, or at least three years.

In particular embodiments, the additional therapeutic agent is an anti-VEGF agent or anti-PDGF agent, e.g., ranibizumab, bevacizumab, SFlt01 or aflibercept. In particular embodiments, the additional therapeutic agent is an anti-tumor agent or an anti-inflammatory agent.

In certain embodiments, the additional therapeutic agent is a viral vector or virion comprising a polynucleotide sequence that encodes the additional therapeutic agent, i.e., an additional viral vector or virion. In certain embodiments, the additional viral vector or virion comprises a capsid protein, e.g., VP1, that is not the same as the modified capsid protein, e.g., VP1, present in the viral vector or virion of the present invention. Such methods may be used to reduce the possibility of an undesirable immune response when a subject has developed an innate or adaptive immune responses against viral particles or capsid proteins therein following administration of the first viral vector or virion (whether that is a viral vector or virion of the present invention or the additional viral vector or virion), such that a second administration of the same viral vector or capsid protein would result in an undesired immune response in the subject. In certain embodiments, a subject is administered an AAVShH10/7m8 viral vector described herein in combination with a different viral vector, e.g., AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV rh.10, avian AAV, bovine AAV, canine AAV, equine AAV, primate AAV, non-primate AAV, bovine AAV, AAV.7m8, AAVShH10, AAV2.5T, AAV2.5T/7m8, AAV9/7m8, or AAV5/7m8. In certain embodiments, the subject is administered an AAVShH10/7m8 viral vector described herein in combination with an AAV2 vector or a variant thereof, e.g., for treating an ocular disease or disorder. In certain embodiments, a subject is administered an AAVShH10/7m8 viral vector described herein in combination with an AAVrh10 vector, e.g., for expressing one or more therapeutic proteins in the liver.

In certain embodiments, the present invention includes a method of treating or preventing a disease or disorder in a subject in need thereof, comprising administering to the subject a first pharmaceutical composition comprising a pharmaceutically acceptable excipient and a first recombinant virus or viral vector, said first virus or vector comprising: (a) a first modified capsid protein, wherein the first modified capsid protein is a modified AAVShH10, AAV1, or AAV6 capsid protein comprising a peptide insertion relative to a corresponding parental AAVShH10, AAV1, or AAV6 capsid protein, wherein the peptide insertion comprises the amino acid sequence LGETTRP (SEQ ID NO:6), and wherein the insertion site is located between amino acid residues 456 and 457, amino acid residues 457 and 458, or amino acid residues 458 and 459 of VP1 of the AAVShH10 capsid protein or the corresponding residues of the AAV1 or AAV6 capsid protein, and (b) a first polynucleotide sequence that encodes a first therapeutic gene product; in combination with a second pharmaceutical composition comprising a pharmaceutically acceptable excipient and a second recombinant virus or viral vector, said second virus or vector comprising: (a) a second modified capsid protein, wherein the modified capsid protein is not the modified AAVShH10, AAV1, or AAV6 capsid protein, and (b) a second polynucleotide sequence that encodes a second therapeutic gene product. In certain embodiments, the second modified capsid protein is an AAV2 capsid protein, or a modified AAV2 capsid protein, optionally an AAV2.7m8 capsid protein. The first and second therapeutic gene products are the same or different. In certain embodiments, the disease or disorder is an ocular disease or disorder, and the first and second pharmaceutical compositions are administered to the eye, e.g., intravitreally. In particular embodiments, one or both of the first and second therapeutic gene products is an anti-vascular endothelial growth factor (anti-VEGF) agent. In particular embodiments, the disease or disorder is selected from the group consisting of: age-related macular degeneration (AMD), wet-AMD, dry-AMD, retinal neovascularization, choroidal neovascularization, diabetic retinopathy, proliferative diabetic retinopathy, retinal vein occlusion, central retinal vein occlusion, branched retinal vein occlusion, diabetic macular edema, diabetic retinal ischemia, ischemic retinopathy, and diabetic retinal edema.

In some embodiments, the disease or disorder is a liver disease or disorder, and the first and second pharmaceutical compositions are administered parenterally, optionally intravenously. In various embodiments, the first pharmaceutical composition and the second pharmaceutical composition are administered sequentially in either order, wherein a period of time passes between the sequential administrations. For example, the period of time may be at least one month, at least 3 months, at least 6 months, at least one year, at least 18 months, at least two years, or at least three years. In particular embodiments, one or both of the first and second therapeutic gene products is alpha-1 antitrypsin, factor IX, factor VIII, C1-esterase inhibitor, β-globin or γ-globin. In certain embodiments, the disease or disorder is selected from the group consisting of: alpha-1 antitrypsin deficiency, hemophilia B, hemophilia A, hereditary angioedema, or β-thalassemia.

In some embodiments, the subject methods result in a therapeutic benefit, e.g. preventing the development of a disorder, halting the progression of a disorder, reversing the progression of a disorder, etc. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy.

In some instances, the expression of the transgene, e.g. as detected by measuring levels of gene product, by measuring therapeutic efficacy, etc., may be observed two months or less after administration, e.g. 4, 3 or 2 weeks or less after administration, for example, 1 week after administration of the subject composition. Expression of the transgene is also expected to persist over time. Accordingly, in some instances, the expression of the transgene, e.g. as detected by measuring levels of gene product, by measuring therapeutic efficacy, etc., may be observed 2 months or more after administration of the subject composition, e.g., 4, 6, 8, or 10 months or more, in some instances 1 year or more, for example 2, 3, 4, or 5 years, in certain instances, more than 5 years.

In particular embodiments, a subject is administered to one eye or to each of both eyes about 1×10⁸ vector genomes or more, in some cases 1×10⁹, 1×10¹⁰, 1×10¹¹, 1×10¹², or 1×10¹³ vector genomes or more, in certain instances, 1×10¹⁴ vector genomes or more. In some cases, the amount of vector genomes that is delivered is at most about 1×10¹⁵ vector genomes, e.g. 1×10¹⁴ vector genomes or less, for example 1×10¹³, 1×10¹², 1×10¹¹, 1×10¹⁰, or 1×10⁹ vector genomes or less, in certain instances 1×10⁸ vector genomes, and sometimes no less than 1×10⁸ vector genomes. In some cases, the amount of vector genomes that is delivered is 1×10¹⁰ to 1×10¹¹ vector genomes. In some cases, the amount of vector genomes that is delivered is 1×10¹⁰ to 3×10¹² vector genomes. In some cases, the amount of vector genomes that is delivered is 1×10⁹ to 3×10¹³ vector genomes. In some cases, the amount of vector genomes that is delivered is 1×10⁸ to 3×10¹⁴ vector genomes.

In some cases, the amount of pharmaceutical composition to be administered may be measured using multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵-1×10⁷. In some cases, the MOI may be 1×10⁴ to 1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹ 1×10¹² 1×10¹³ 1×10¹⁴ 1×10¹⁵ 1×10¹⁶, 1×10¹⁷, and 1×10¹⁸ MOI.

In some aspects, the amount of pharmaceutical composition comprises about 1×10⁸ to about 1×10¹⁵ particles of recombinant virions or viruses, about 1×10⁹ to about 1×10¹⁴ particles of recombinant virions or viruses, about 1×10¹⁰ to about 1×10¹³ particles of recombinant virions or viruses, or about 1×10¹¹ to about 3×10¹² particles of recombinant virions or viruses.

In some aspects, no virion or vector is detected in the human subject's tear, blood, saliva or urine samples 7, 14, 21 or 30 days after administering said pharmaceutical composition. In some aspects, the presence of the viral vector is detected by qPCR or ELBA as known in the art.

In some aspects, a subject's best corrected visual acuity (BCVA) improves by 1, 2 3, 4, 5 or more lines following a method of treatment described herein.

In some aspects, a reduction in neovascularization as assessed by Fluorscein Angiography (FA) follows the administering step.

In some cases, retinal thickness may be measured to examine the effects of treatment. In some cases, the central retinal thickness of the human subject does not increase by more than 50 microns, 100 microns, or 250 microns within 12 months following treatment with the pharmaceutical composition of the disclosure. In some cases, the central retinal thickness of the human subject decreases by at least 50 microns, 100 microns, 200 microns, 250 microns, 300 microns, 400 microns, 500 microns, 600 microns within 3 months, 6 months or 9 months 12 months following treatment with the pharmaceutical composition of the disclosure. The decrease in the central retinal thickness of the human subject may be measured comparing the central retinal thickness at point in time to a baseline measurement taken at or within 1, 3, 7 or 10 days of the administration of the pharmaceutical composition of the disclosure.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1

### Construction of ShH10 Mutants

Site-directed mutagenesis and recombinant DNA techniques were used to generate variants of ShH10, which included a 7m8 insert consisting of LALGETTRPA (SEQ ID NO:14) in the ShH10 VP1 capsid protein, between amino acid residues 456 and 457 (“ShH10/7m8(457)”), between amino acid residue 457 and 458 (“ShH10/7m8(458)”), or between amino acid residues 458 and 459 (“ShH10/7m8(459)”).

The ShH10 variants were generated by triple transfection of cells with: a first plasmid comprising an expression cassette with ITRs flanking a transgene (e.g., GFP or luciferase); a second plasmid encoding the Rep/Cap genes; and a third plasmid containing adenovirus-helper functions, followed by ultracentrifugation to separate the empty and full capsids.

The ShH10 variant viruses were characterized by quantitative PCR to establish titer and packaging, and western blot was conducted to ensure the correct ratio of the VP1, VP2 and VP3 capsid proteins. All ShH10/7m8 variants packaged, resulting in high titers (˜1E14 vg/ml), and western blots showed the correct ratio of VP1, VP2 and VP3.

### Example 2

### Transduction of HEK293, U87 and HepG2 Cells

In vitro transductions were performed on HEK293, U87 and HepG2 cells at an MOI of 3×10⁵ for five days. Images were captured and flow cytometry was performed at the end of the study in order to assess the percentage of transduced cells and the median fluorescence intensity.

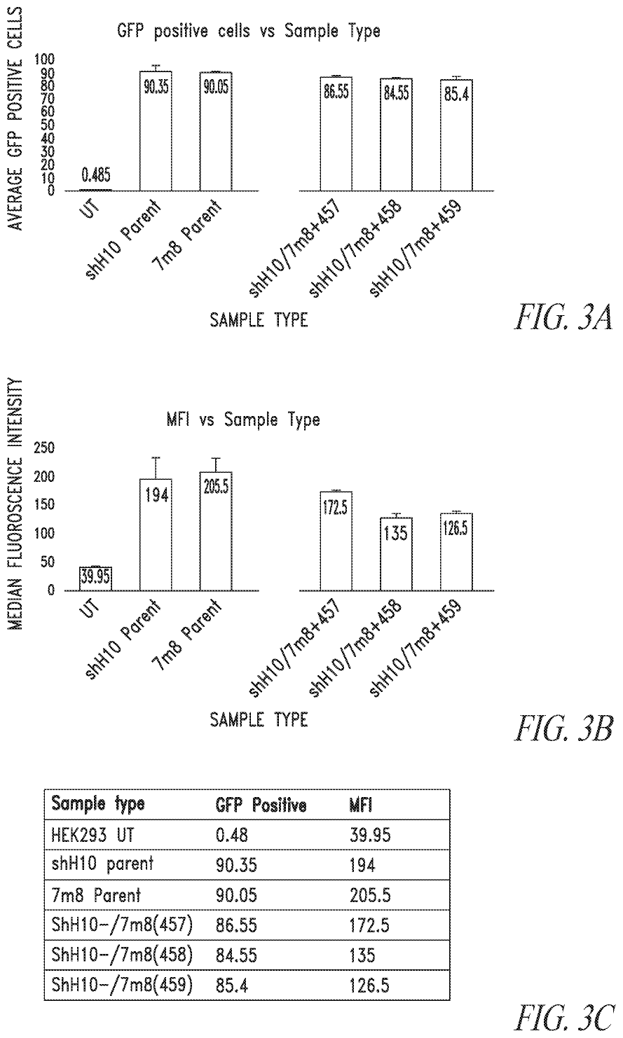

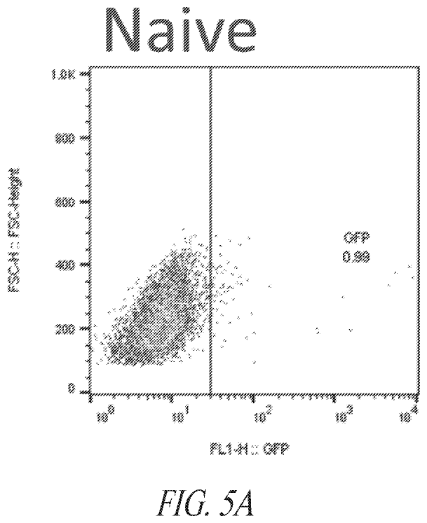

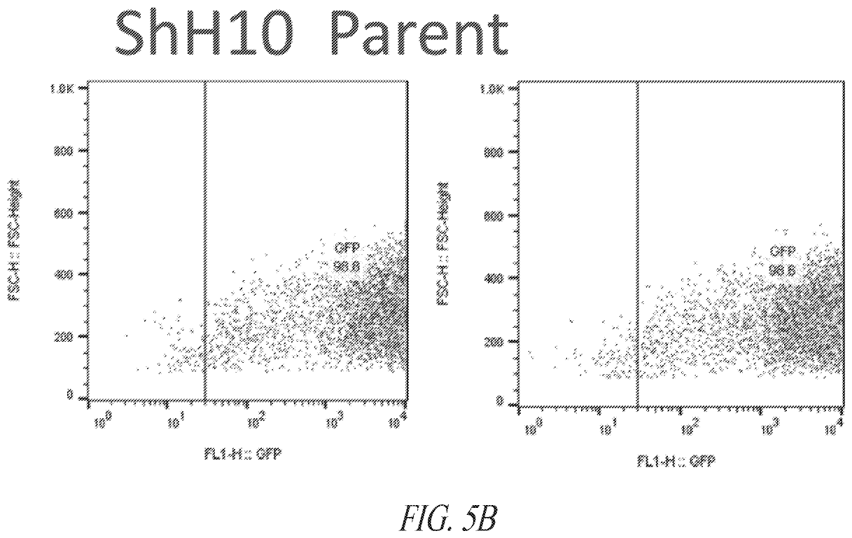

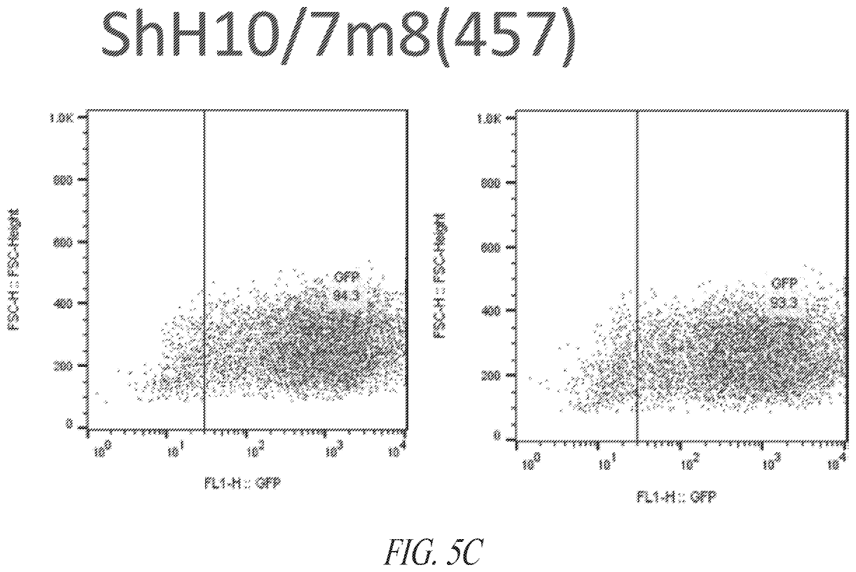

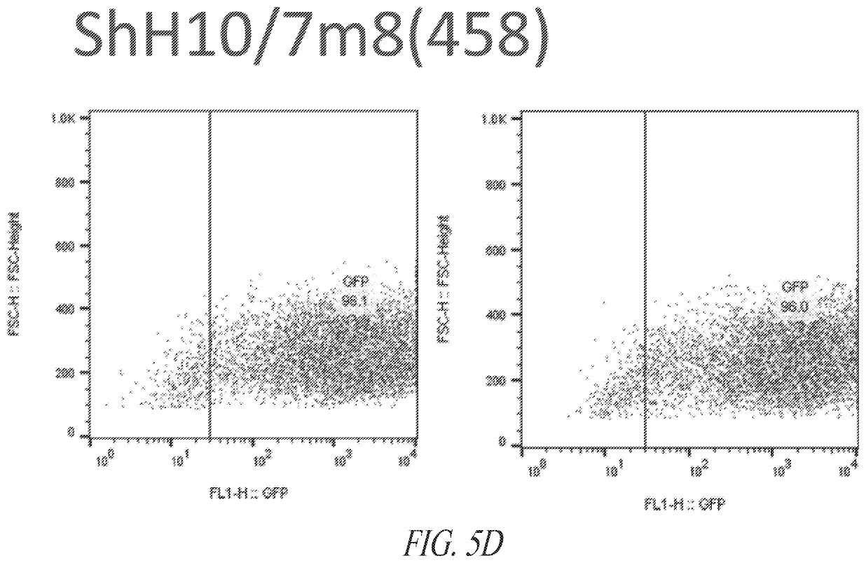

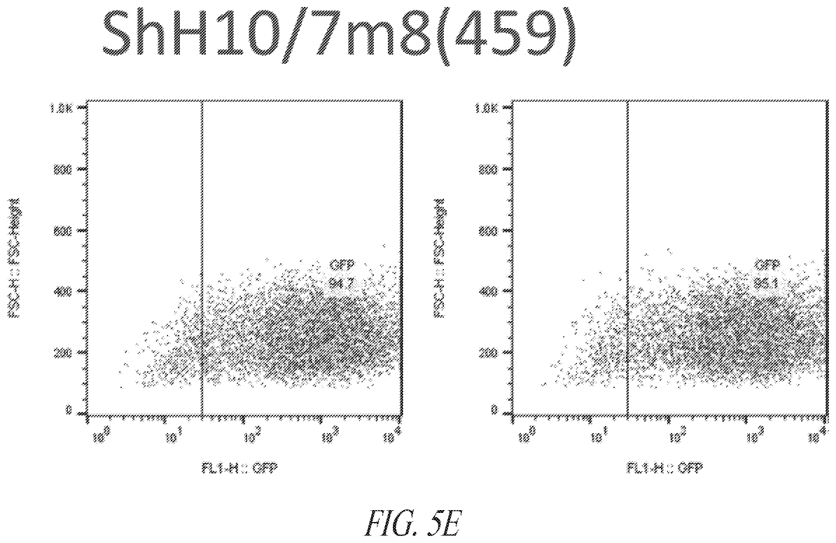

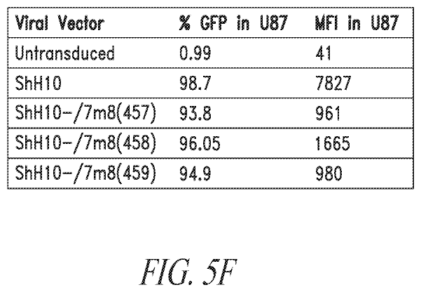

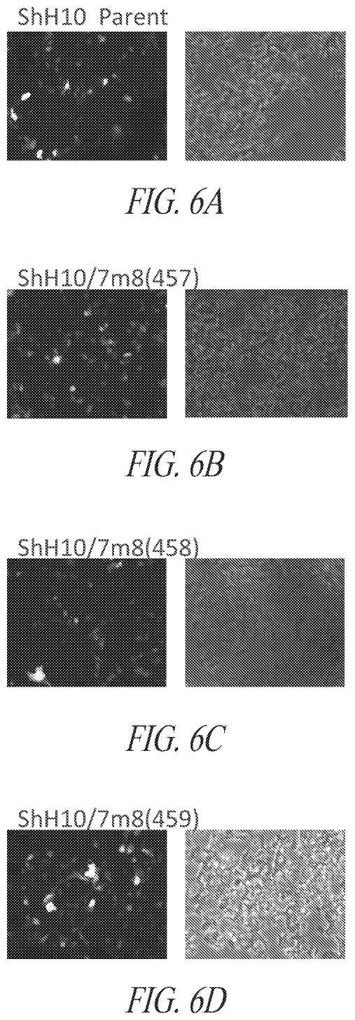

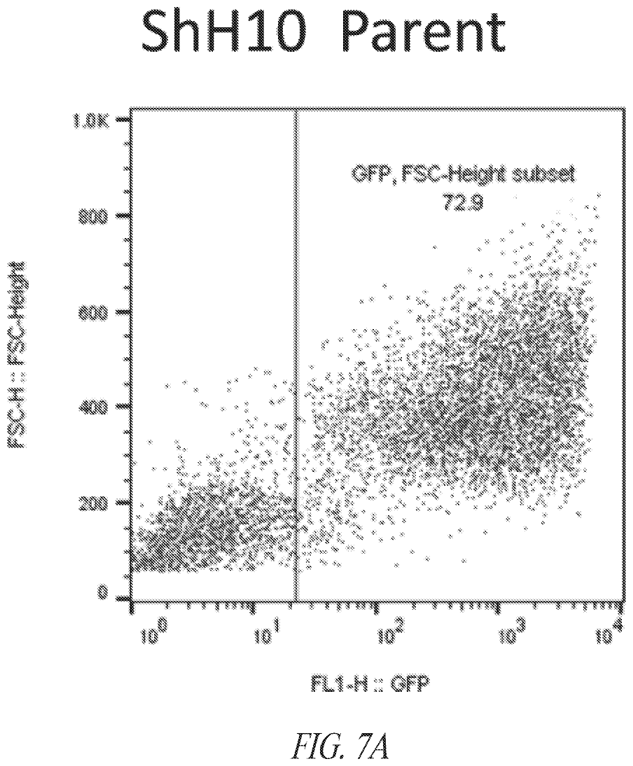

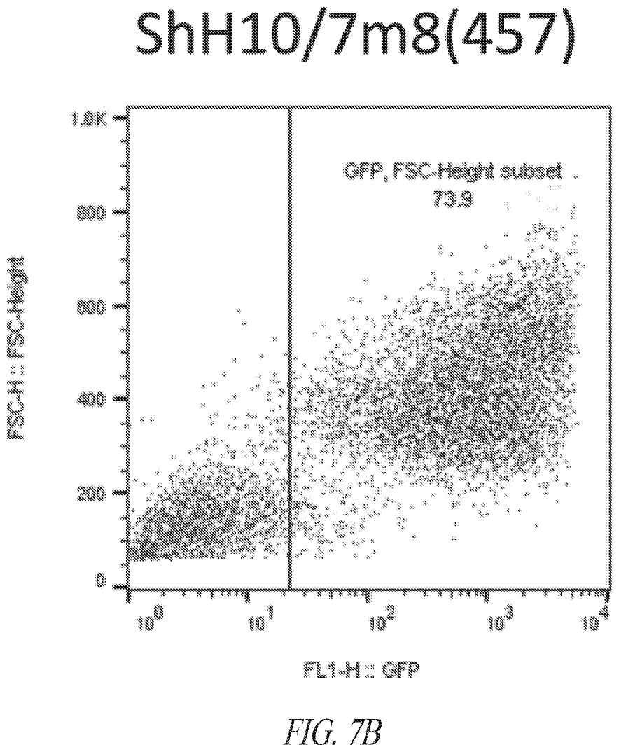

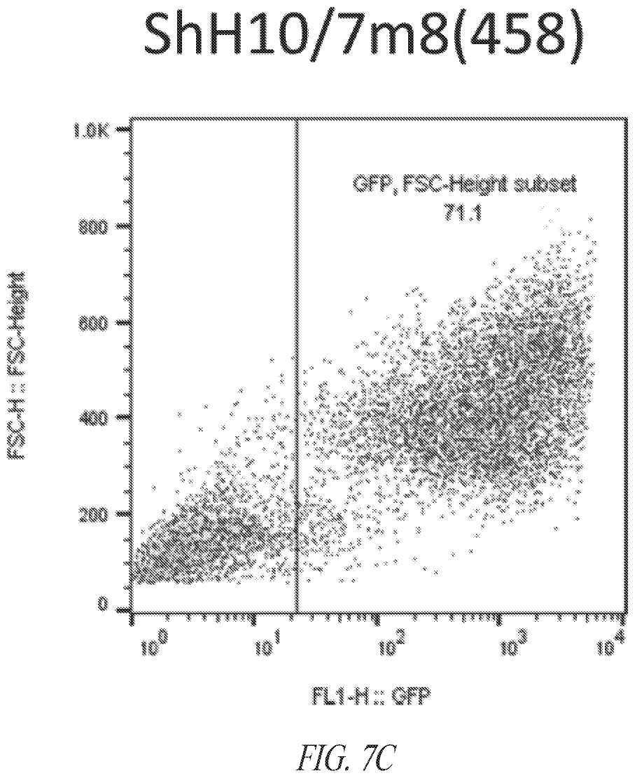

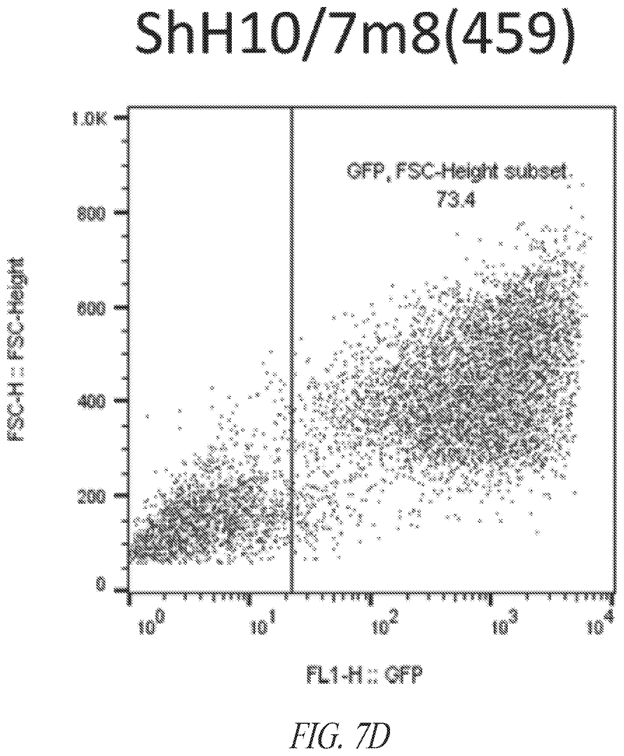

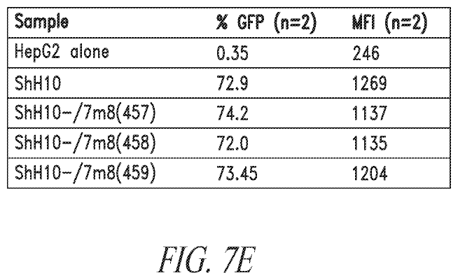

ShH10/7m8 variants transduced HEK293, HepG2, and U87 cells at levels similar to parent ShH10. The results of these studies are shown in FIGS. 1-3 (HEK293 cells), FIGS. 4-5 (U87 cells), and FIGS. 6-7 (HepG2 cells).

### Example 3

### Heparan Binding Affinity of AAVShH10/7m8

The ability of the AAVShH10/7m8 variants ShH10/7m8(457), ShH10/7m8(458), and ShH10/7m8(459) to bind HSPG was determined by performing a heparan binding assay using a pre-packed GE heparan column. Vector was loaded on the column, which was then washed, and finally eluted with increasing concentrations of NaCl (100 mM to 1 M). Fractions of Load, Flow-through, Wash, and Elution were collected and analyzed by dot-blot using the B1 antibody, as outlined in FIG. 8A.

The AAVShH10/7m8 variants demonstrated similar levels of binding affinity to the heparan column as AAV.7m8 (7m8) and parental ShH10. FIGS. 8B-8F show the binding elution profile as determined by dot-blot for control AAV.7m8 and ShH10 (FIGS. 8B and 8C) as well as ShH10/7m8(457) (FIG. 8D), ShH10/7m8(458) (FIG. 8E), and ShH10/7m8(459) (FIG. 8F).

### Example 4

### Neutralizing Antibody Profile Using IVIG

A 3-fold dilution series of intravenous immunoglobulin (IVIG) was mixed with each ShH10/7m8 variant and then added onto 293T cells. These were incubated for 3 days followed by measurement of transgene expression (GFP) using a plate reader. IC₅₀ value was generated at the level when 50% inhibition of transgene expression was observed in presence of IVIG.

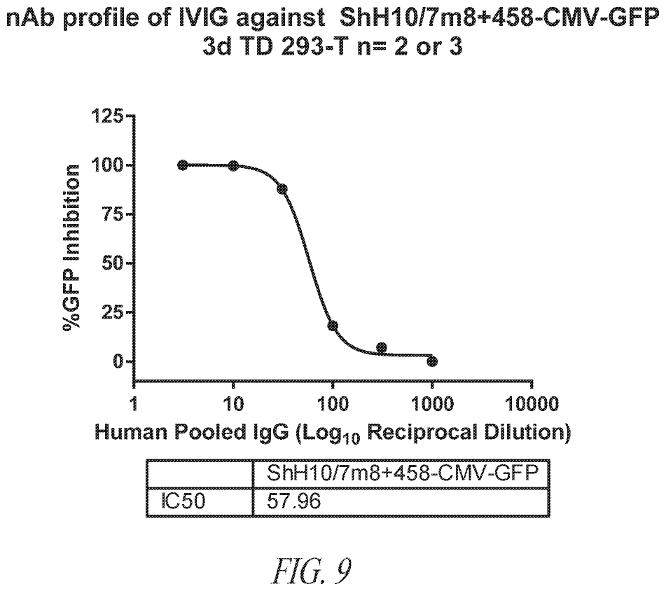

FIG. 9 shows IC₅₀ value of ShH10/7m8(458) at ˜58, which is better than what was observed with AAV.7m8 (˜125) (data not shown).

### Example 5

### Expression and Tropism in Pig Retinal Explants

Ex vivo pig retinal explants maintained on trans-wells were transduced with the parental ShH10 virus, or variant virus ShH10/7m8(457), each expressing GFP, at an MOI of 4×10⁴. Two weeks post transduction, explants were cryosectioned, and probed to detect Rhodopsin (to detect rod cells), GFAP (to detect Muller cells), TuJ1 (to detect retinal ganglion cells), CHX10 (to detect bipolar cells), and GFP. Immunofluorescent images were captured for ShH10/7m8(457) (FIGS. 10A-10L) and ShH10 (FIGS. 10M-10P).

ShH10/7m8(457) transduced the explants better than ShH10 parent. Various cell layers were transduced by this variant. In particular, high levels of GFP expression mediated by transduction of Shh10/7m8(457) was observed in Muller glia and photoreceptors. Expression was also observed in retinal ganglion cells and bipolar cells.

### Example 6

### In Vivo Expression in Gerbil Retina

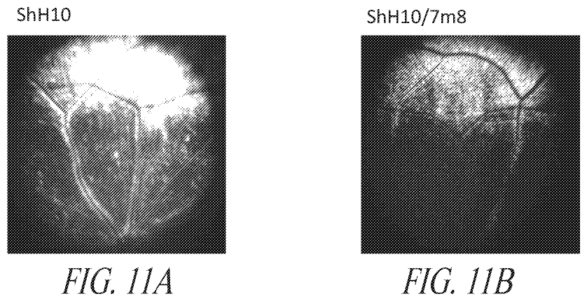

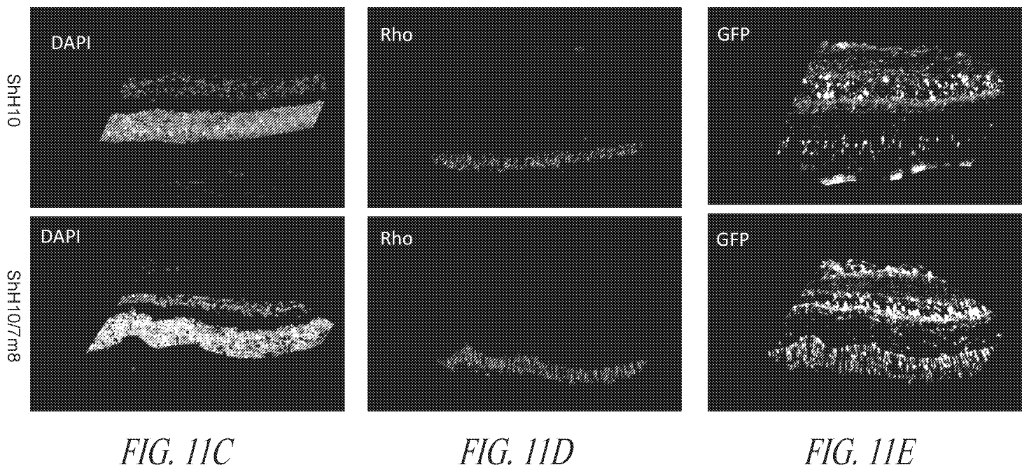

Gerbils were intravitreally (IVT) injected with either ShH10 or ShH10/7m8(457) expressing GFP at 2×10¹⁰ vg/eye. Fundus images were captured at various time-points, including week 12. Data at week 12 showed high levels of transgene expression from parent ShH10 (FIG. 11A) as well as ShH10/7m8(457) (FIG. 11B). After sacrifice, the gerbil retina was isolated and used for immunofluorescent labelling to determine cells that were transduced. Immunofluorescence images show various retinal cell types expressing GFP, including photoreceptors, outer and inner nuclear layers, and RGCs (FIG. 11C-11E).

### Example 7

### In Vivo Expression in African Green Monkey Retina

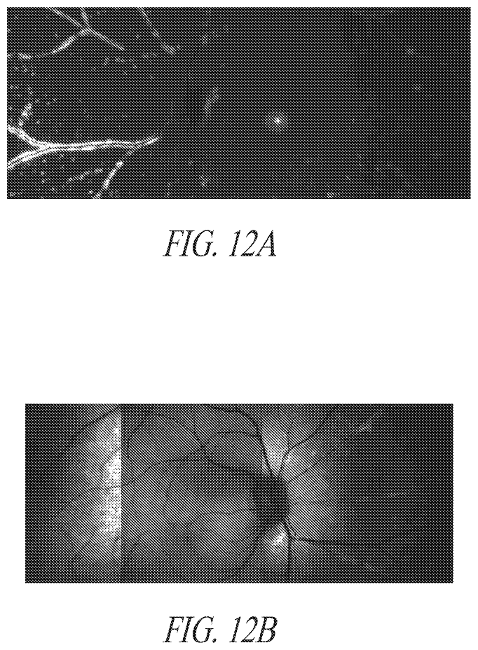

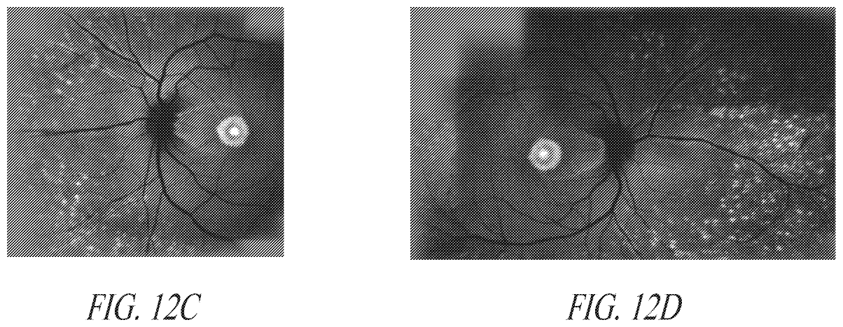

African green monkeys received intravitreal injections of 2×10¹² vg/eye of ShH10/7m8(457) or ShH10 expressing GFP. At 4, 8, and 12 weeks post transduction, OCT images were captured using a Heidelberg Spectralis machine. Week 12 images for ShH10 and ShH10/7m8(457) are presented in FIG. 12A-12B and FIG. 12C-12D, respectively. Based on a visual assessment of the images, ShH10/7m8(457) appeared to mediate higher level of transduction leading to more transgene expression.

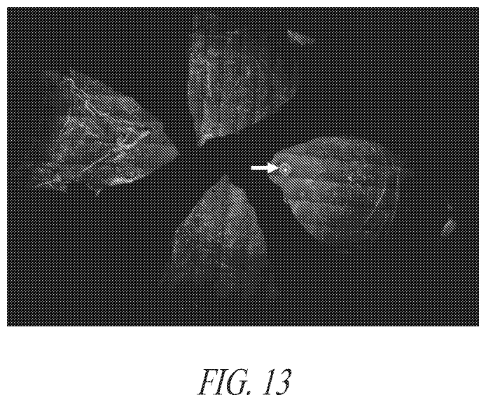

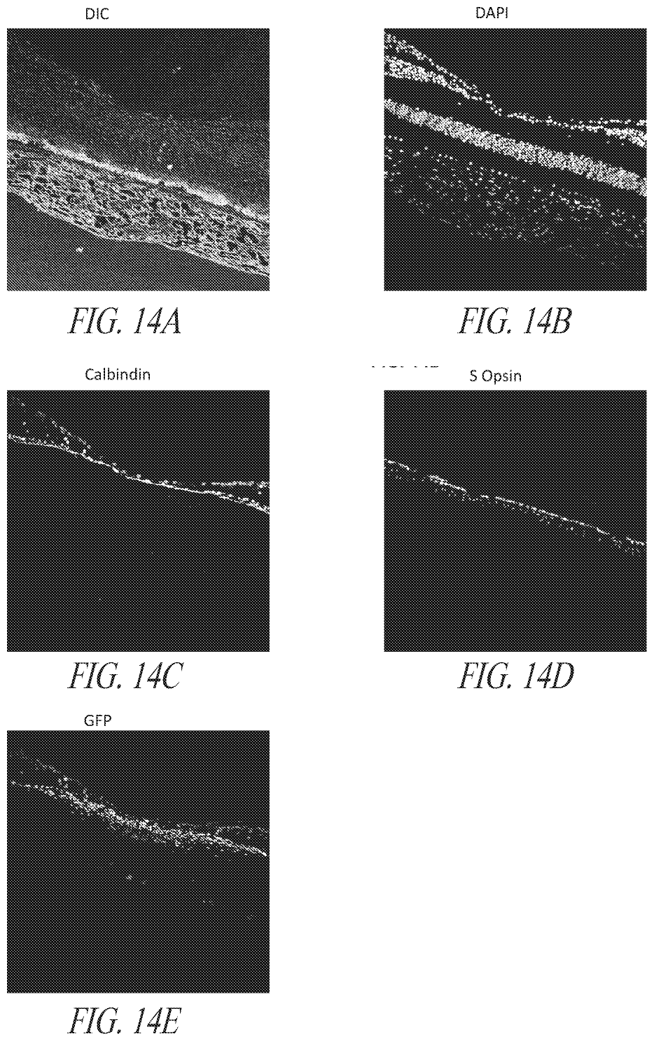

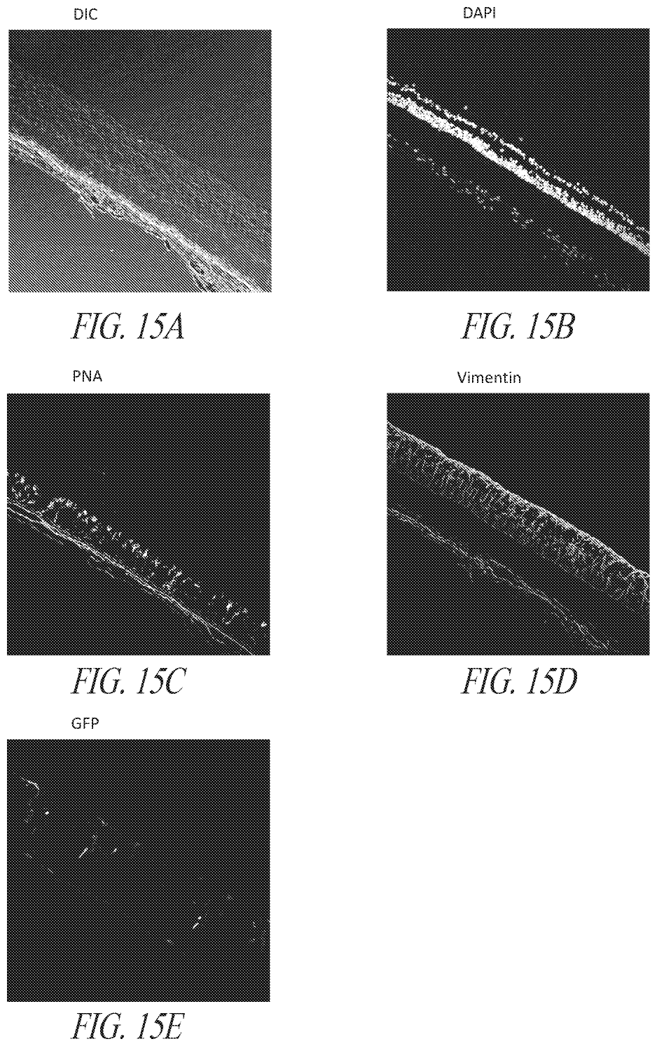

After 12 weeks, monkeys were sacrificed, and retinas were removed. FIG. 13 provides a live fluorescence image of the flat-mounted retina of a monkey transduced with ShH10/7m8(457). GFP expression is evident both in the fovea and the periphery of the non-human primate retina. Following live imaging, the retina was cryosectioned, and sections from the fovea and periphery probed for DAPI (to detect cell nuclei) (FIGS. 14B, 15B), calbindin (to detect bipolar cells) (FIG. 14C), s-opsin (to detect s-cones) (FIG. 14D), PNA (to detect cone cells) (FIG. 15C), vimentin (to detect Muller cells) (FIG. 15D), and GFP (FIGS. 14E, 15E). GFP expression colocalized primarily with calbindin, and to a lesser extent with S Opsin in the fovea indicating transduction of bipolar cells and s-cones along with Muller glia in that region, and primarily with vimentin and to a lesser extent with PNA in the periphery, indicating transductions of Muller cells and cones.

### Example 8

### In Vivo Expression in Mice Following Systemic Delivery

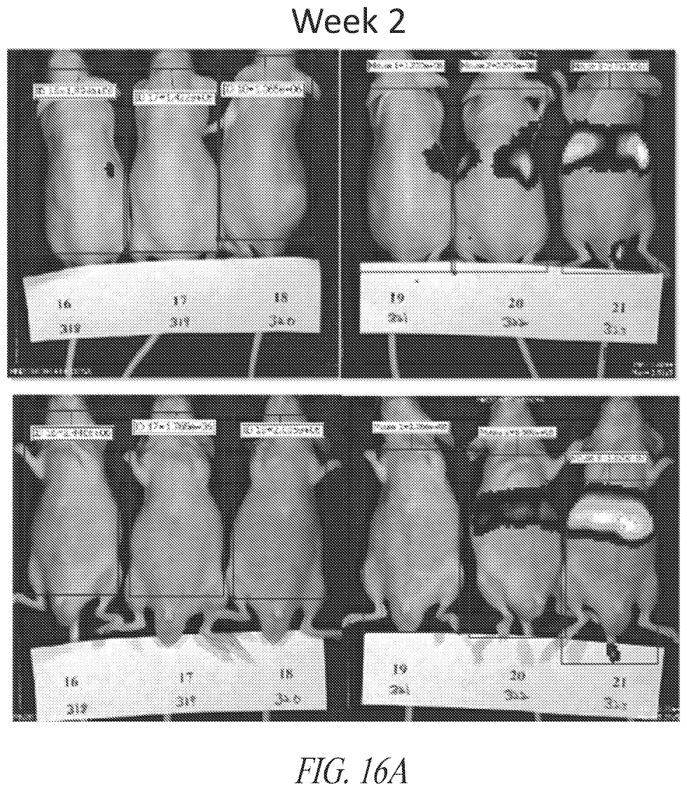

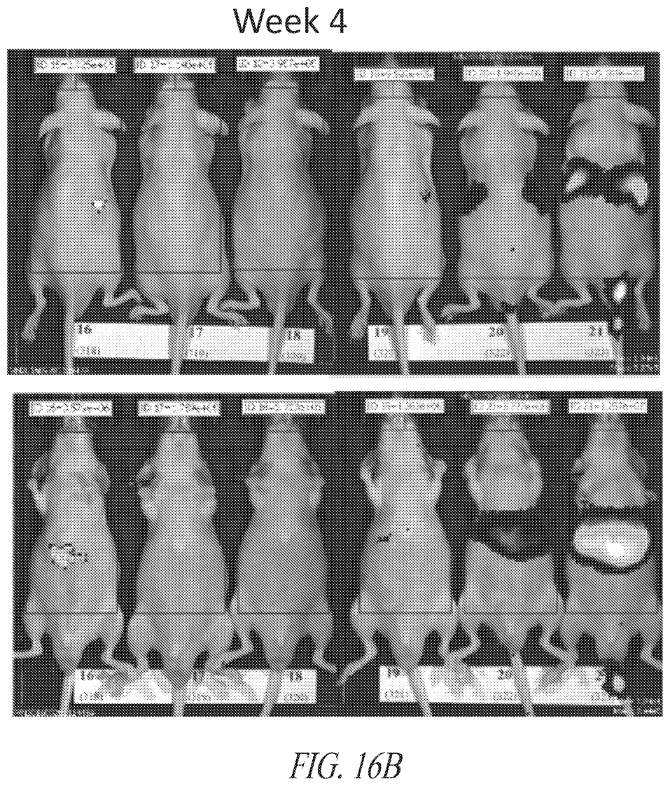

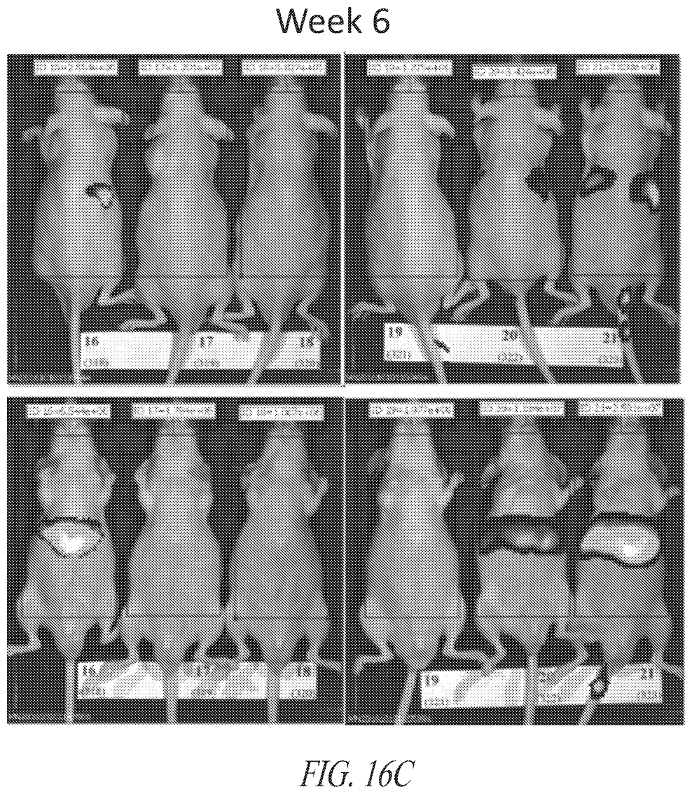

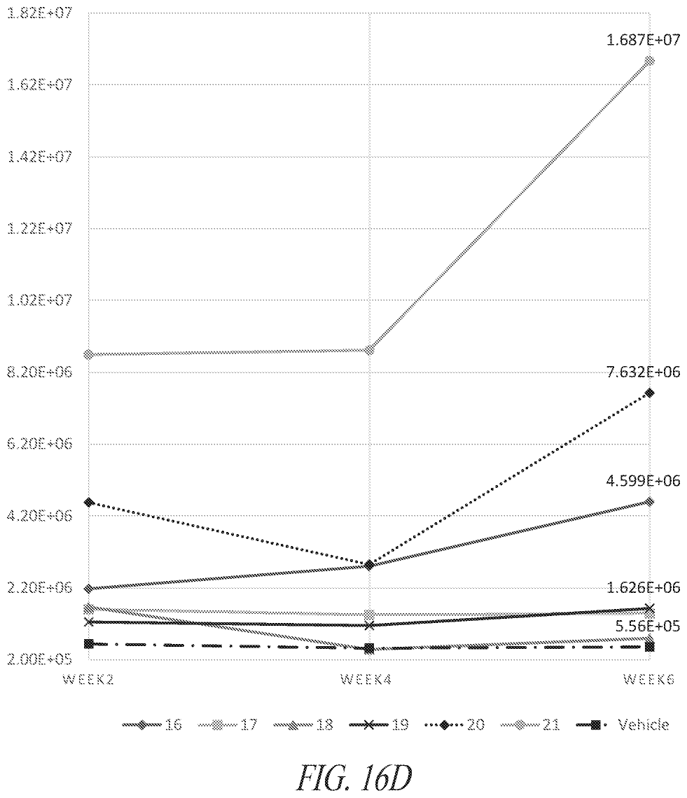

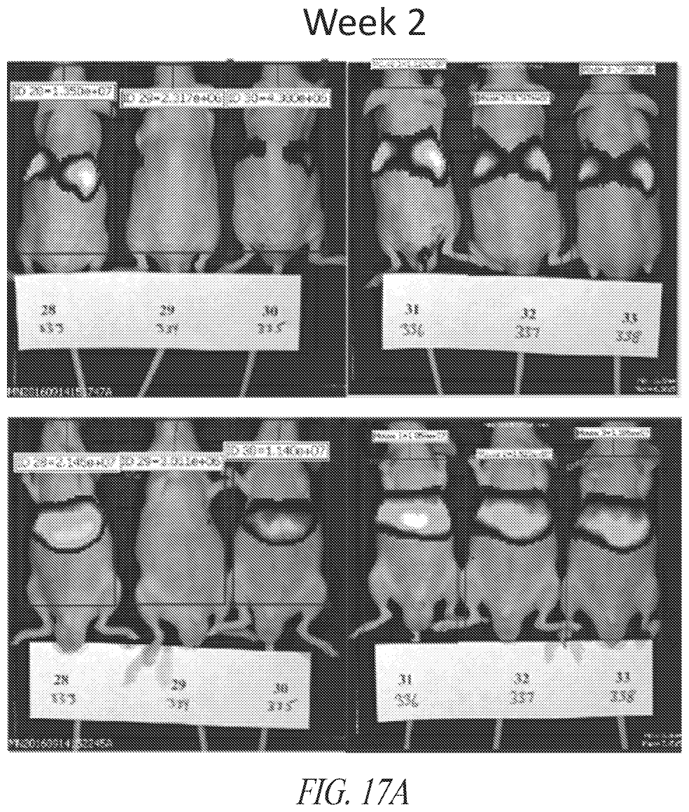

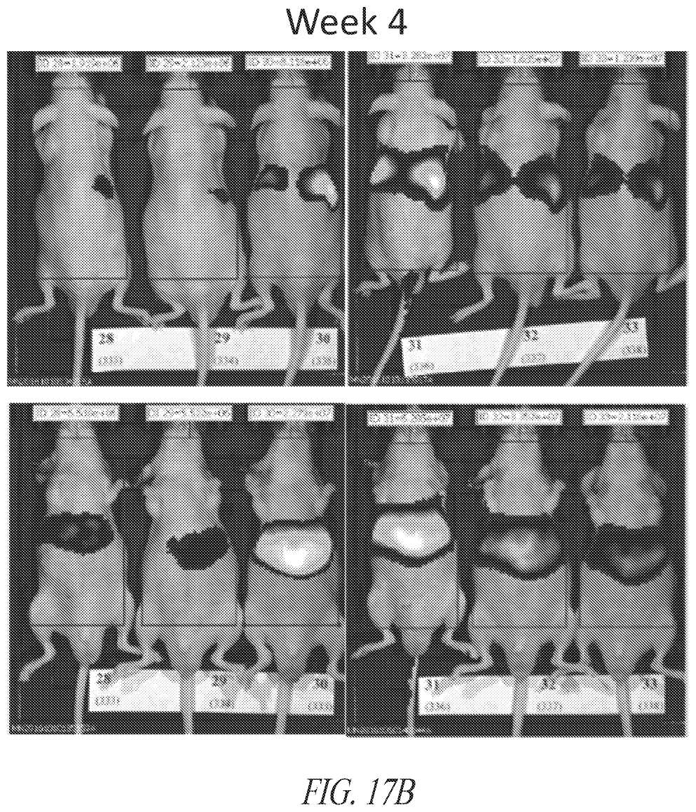

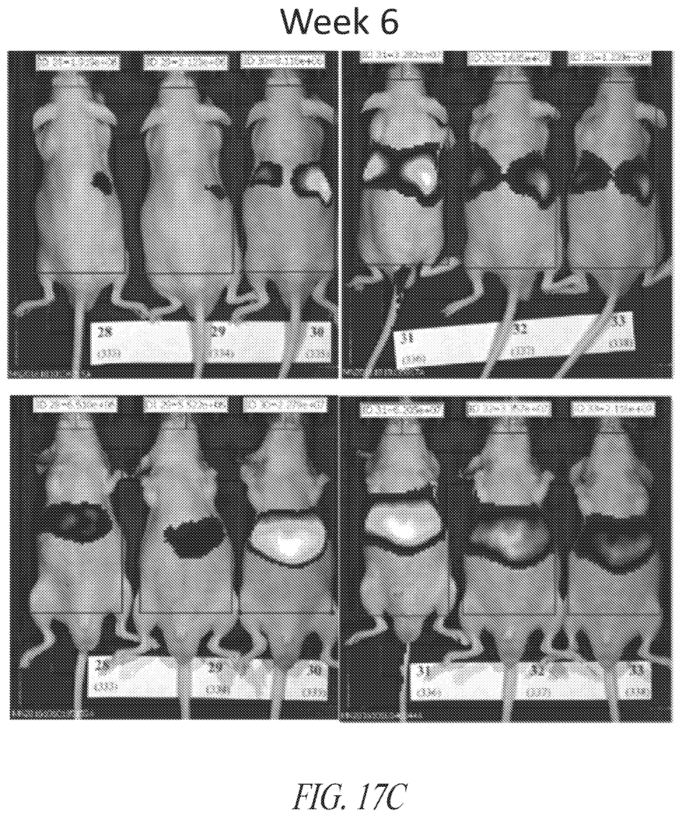

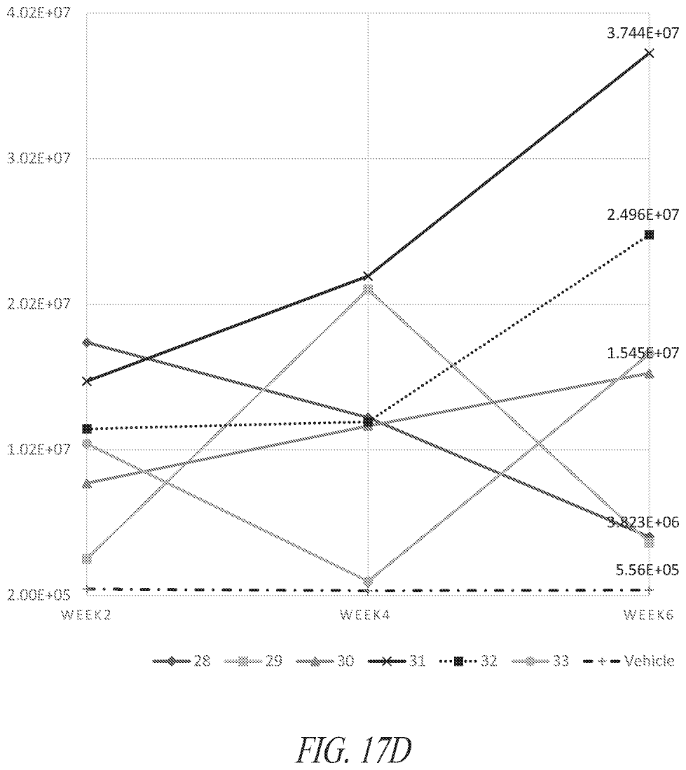

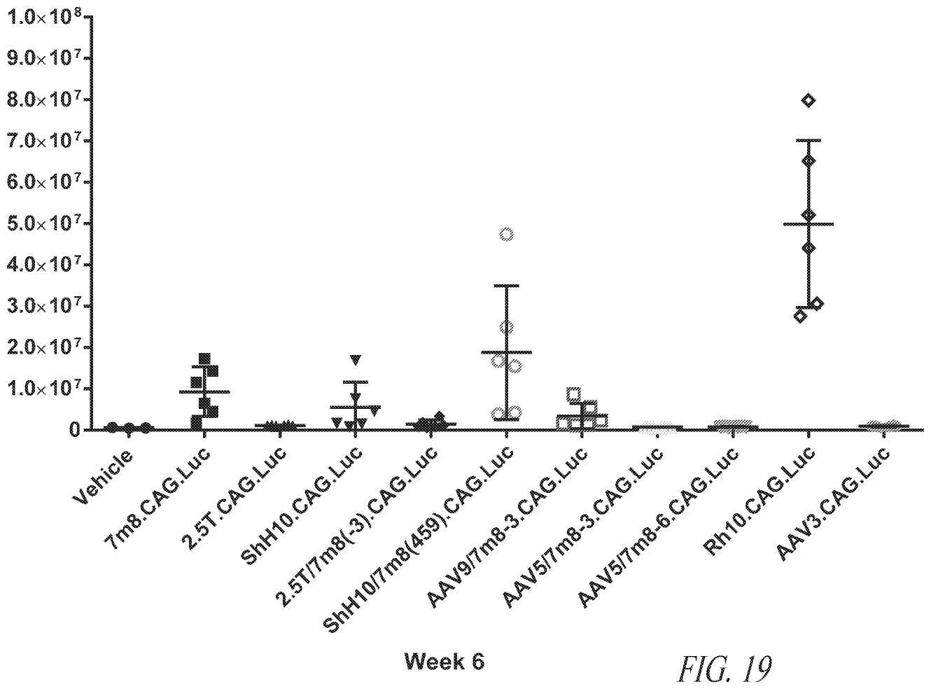

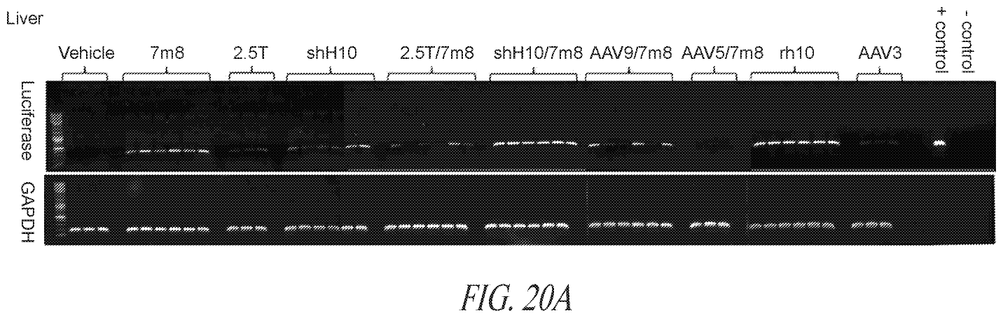

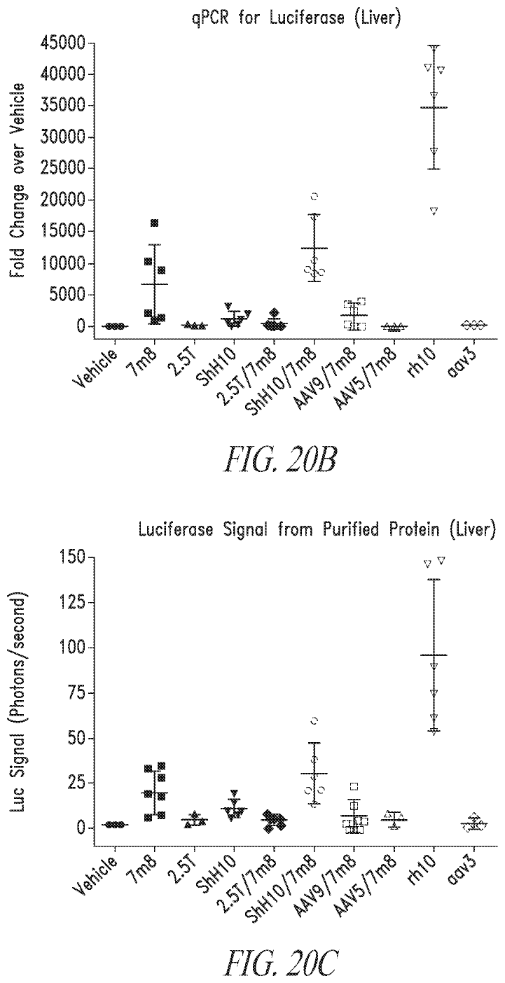

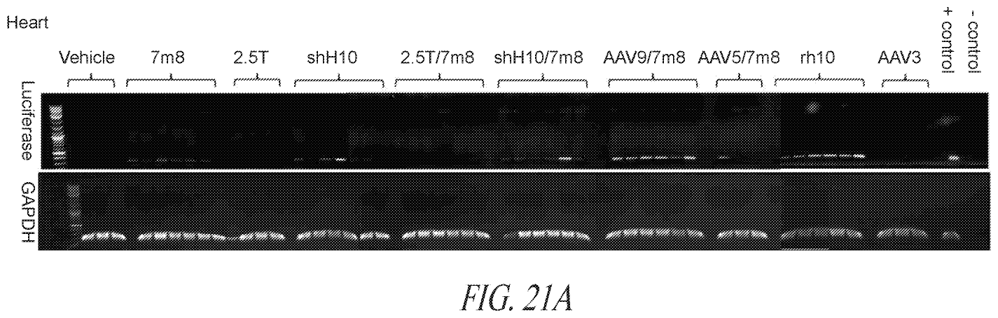

Male, hairless SKH-1 mice were intravenously injected with a dose of 1×10¹¹ vg of one of the following viruses: AAV.7m8, AAV2.5T, ShH10, 2.5T/7m8(−3), ShH10/7m8(458), AAV9/7m8, AAV5/7m8, AAVrh10, or AAV3. Each vector expressed luciferase driven by the ubiquitous CAG promoter. In vivo live imaging was performed using the IVIS Spectrum at weeks 2, 4, and 6 to assess luciferase expression kinetics. IVIS images of mice treated with ShH10 and ShH10/7m8(458) are depicted in FIGS. 16A-16C and 17A-17C, respectively, and presented as graphs in FIGS. 16D and 17D respectively. Total luciferase expression based on IVIS imaging at six weeks post transduction for each virus is depicted in FIG. 19. Animals were sacrificed at week 6 and blood, liver, heart, brain, lungs, spleen, pancreas, kidneys, quadriceps, and gonads were collected using ultra-clean procedure. Tissues such as liver, brain, and heart were analyzed using reverse transcriptase quantitative PCR (RT-qPCR) to determine levels of luciferase mRNA, and finally luciferase activity levels were assessed in protein extracts.

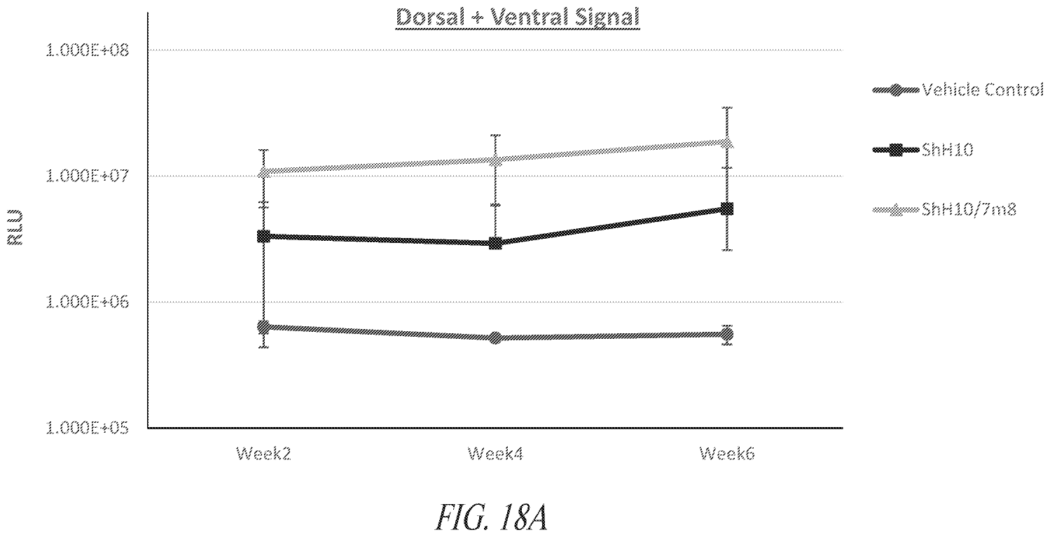

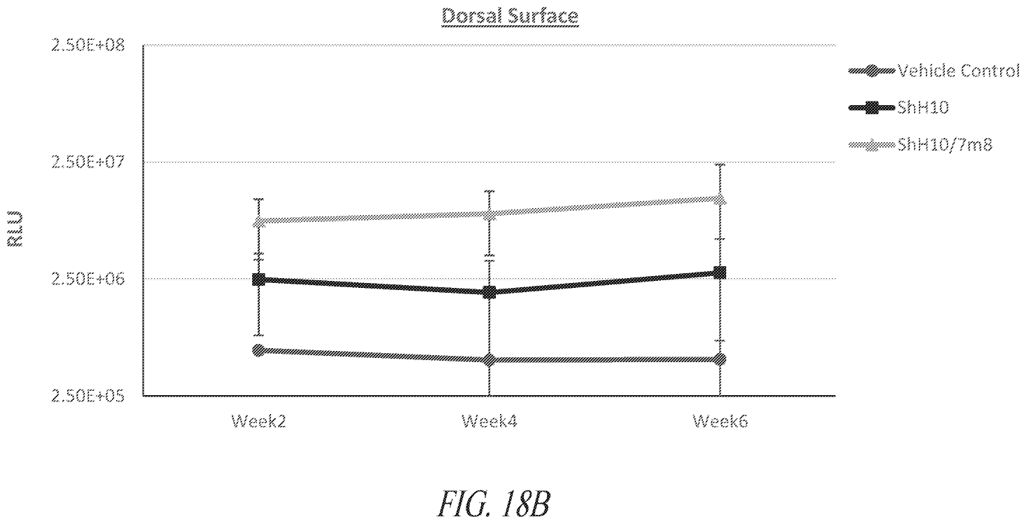

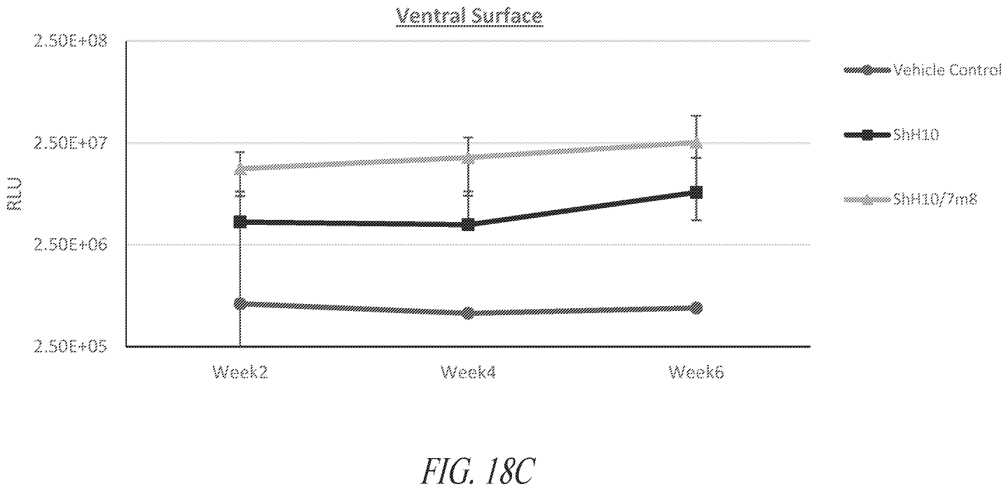

IVIS data at various time-points show ShH10/7m8(458) mediates about 4-fold higher levels of luciferase expression compared to ShH10 parent capsid (FIGS. 18A-18C). For example, the average expression at week 6 was 5.5×10⁶ RLU for ShH10, versus 1.9×10⁷ RLU for ShH10/7m8(458).

mRNA levels of luciferase transgene as well as luciferase protein expression in liver was ˜4-fold higher from ShH10/7m8(458)-mediated vector as compared to ShH10 (FIGS. 20A-20C).

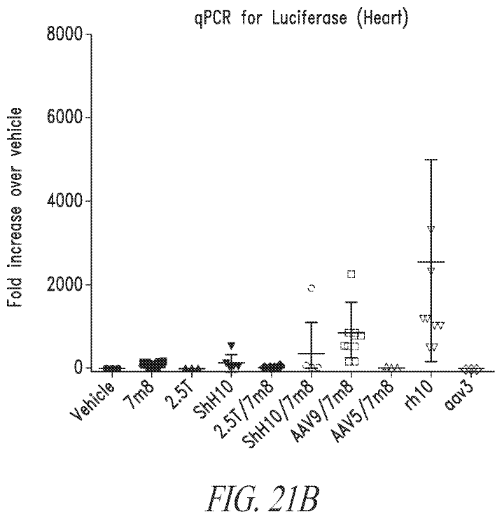

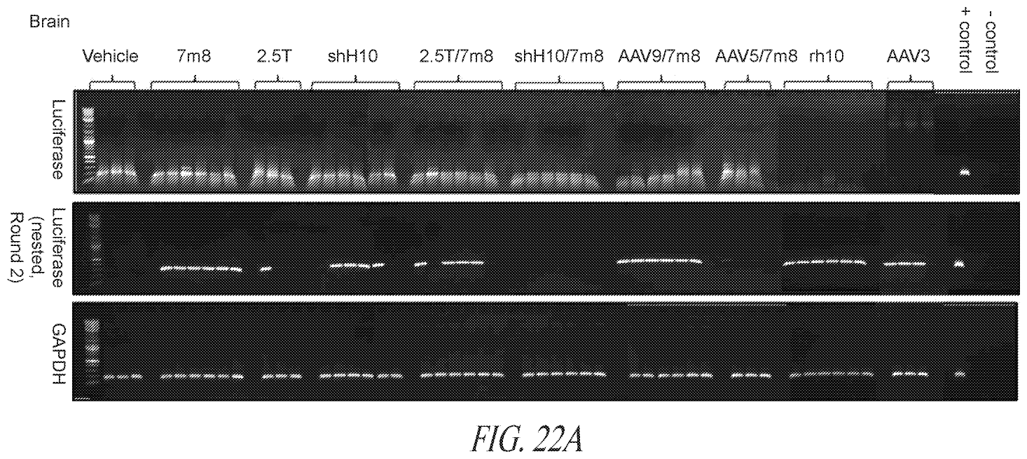

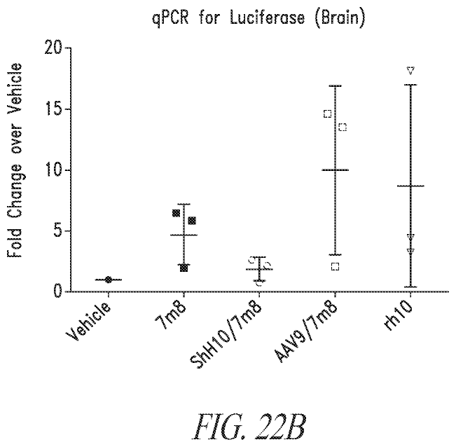

Extremely low, to no expression of transgene mediated by ShH10/7m8(458) was observed in any other tissues analyzed as compared to luciferase expression mediated by ShH10 (FIGS. 21A-21B and 22A-22B). This unexpectedly shows that ShH10/7m8(458) is more tissue specific than the parental ShH10, particularly for liver.

1. A non-naturally-occurring modified adeno-associated virus (AAV) capsid protein comprising a peptide insertion relative to a corresponding parental AAV capsid protein, wherein the peptide insertion comprises the amino acid sequence LGETTRP (SEQ ID NO:6) or the amino acid sequence LALGETTRPA (SEQ ID NO:14), wherein the insertion site is located between amino acid residues 456 and 457, amino acid residues 457 and 458, or amino acid residues 458 and 459 of VP1 of AAVShH10, or at a corresponding position in the capsid protein of another AAV serotype.

2. The modified AAV capsid protein of claim 1, wherein the AAV is an AAVShH10, AAV1, or AAV6.

3. A polynucleotide comprising a nucleic acid sequence encoding the modified AAV capsid protein of claim 1 or claim 2.

4. An expression vector comprising the polynucleotide of claim 3, wherein the nucleic acid sequence encoding the modified AAV capsid protein is operably linked to a promoter sequence.

5. A cell comprising the expression vector of claim 4.

6. The cell of claim 5, further comprising a polynucleotide that encodes a therapeutic protein.

7. The cell of claim 5 or claim 6, further comprising a polynucleotide that encodes a rep protein.

8. A recombinant virus or viral vector comprising the modified capsid protein of claim 1 or claim 2.

9. The recombinant virus or viral vector of claim 8, wherein the recombinant virus or viral vector is an AAV.

10. The recombinant virus or viral vector of claim 9, wherein the AAV is AAVShH10, AAV1, or AAV6

11. The recombinant virus or viral vector of any of claims 8-10, wherein the recombinant virus is eluted from a heparan column at a salt concentration of about 0.2 M to about 0.4 M.

12. The recombinant virus or viral vector of any of claims 8-11, wherein the recombinant virus or viral vector is capable of binding to and crossing the inner limiting membrane (ILM) when intravitreally injected into a mammal.

13. The recombinant virus or viral vector of any one of claims 8-12, wherein the recombinant virus or viral vector comprises a polynucleotide sequence that encodes a therapeutic gene product.

14. The recombinant virus or viral vector of claim 13, wherein the therapeutic gene product is an anti-vascular endothelial growth factor (anti-VEGF) agent.

15. The recombinant virus or viral vector of claim 13, wherein the therapeutic gene product is alpha-1 antitrypsin, a factor IX, factor VIII, C1-esterase inhibitor, β-globin or γ-globin.

16. The recombinant virus or viral vector of any one of claims 8-15, wherein the recombinant virus or viral vector has an altered cellular tropism as compared to AAVShH10 or AAV6.

17. The recombinant virus or viral vector of any one of claims 8-16, wherein the recombinant virus or viral vector has a greater infectivity of retinal cells or liver cells as compared to AAVShH10, AAV1, or AAV 6.

18. A pharmaceutical composition comprising a pharmaceutically acceptable excipient and the recombinant virus or viral vector of any one of claims 13-17.

19. A method of treating or preventing an ocular disease in a subject in need thereof, comprising administering to the subject by intravitreal injection the pharmaceutical composition of claim 18.

20. The method of claim 19, wherein the recombinant virus or viral vector comprises a modified AAVShH10, AAV1, or AAV6 capsid protein.

21. A method of treating or preventing a disease or disorder in a subject in need thereof, comprising administering to the subject:
(i) a first pharmaceutical composition comprising a pharmaceutically acceptable excipient and a first recombinant virus or viral vector, comprising:
(a) a first modified capsid protein, wherein the first modified capsid protein is a modified AAVShH10, AAV1, or AAV6 capsid protein comprising a peptide insertion relative to a corresponding parental AAVShH10, AAV1, or AAV6 capsid protein, wherein the peptide insertion comprises the amino acid sequence LGETTRP (SEQ ID NO:6), and wherein the insertion site is located between amino acid residues 456 and 457, amino acid residues 457 and 458, or amino acid residues 458 and 459 of VP1 of the AAVShH10 capsid protein or the corresponding residues of the AAV1 or AAV6 capsid protein, and
(b) a first polynucleotide sequence that encodes a first therapeutic gene product; and
(ii) a second pharmaceutical composition comprising a pharmaceutically acceptable excipient and a second recombinant virus or viral vector, comprising:
(a) a second modified capsid protein, wherein the modified capsid protein is not the modified AAVShH10, AAV1, or AAV6 capsid protein, and
(b) a second polynucleotide sequence that encodes a second therapeutic gene product.

22. The method of claim 21, wherein the second modified capsid protein is an AAV2 capsid protein, or a modified AAV2 capsid protein, optionally an AAV2.7m8 capsid protein.

23. The method of claim 21 or claim 22, wherein the first pharmaceutical composition and the second pharmaceutical composition are administered sequentially in either order, and wherein a period of time passes between the sequential administrations.

24. The method of claim 23, wherein the period of time is at least one month, at least 3 months, at least 6 months, at least one year, at least 18 months, at least two years, or at least three years.

25. The method of any of claims 21-24, wherein the first and second therapeutic gene products are the same or different.

26. The method of any of claims 21-25, wherein the disease or disorder is an ocular disease or disorder, and the first and second pharmaceutical compositions are administered intravitreally.

27. The method of claim 26, wherein one or both of the first and second therapeutic gene products is an anti-vascular endothelial growth factor (anti-VEGF) agent.

28. The method of claim 26 or claim 27, wherein the disease or disorder is selected from the group consisting of: age-related macular degeneration (AMD), wet-AMD, dry-AMD, retinal neovascularization, choroidal neovascularization, diabetic retinopathy, proliferative diabetic retinopathy, retinal vein occlusion, central retinal vein occlusion, branched retinal vein occlusion, diabetic macular edema, diabetic retinal ischemia, ischemic retinopathy, and diabetic retinal edema.

29. The method of any of claims 21-25, wherein the disease or disorder is a liver disease or disorder, and the first and second pharmaceutical compositions are administered parenterally, optionally intravenously.

30. The method of claim 29, wherein one or both of the first and second therapeutic gene products is alpha-1 antitrypsin, factor IX, factor VIII, C1-esterase inhibitor, β-globin or γ-globin.

31. The method of claim 29 or claim 30, wherein the disease or disorder is selected from the group consisting of: alpha-1 antitrypsin deficiency, hemophilia B, hemophilia A, hereditary angioedema, or β-thalassemia.


SEQUENCE LISTING (32 sequences)

SEQ ID NO: 1 - protein, 11 aa, Artificial Sequence
  7m8 insersertion sequence motif
  MISC_FEATURE (1)..(2): Xaa is Ala, Leu, Gly, Ser, Thr or absent
  MISC_FEATURE (3)..(6): Xaa is any amino acid
  MISC_FEATURE (10)..(11): Xaa is Ala, Leu, Gly, Ser, Thr or absent
XXXXXXTRPXX

SEQ ID NO: 2 - protein, 11 aa, Artificial Sequence
  7m8 insersertion sequence motif
  MISC_FEATURE (1)..(2): Xaa is Ala, Leu, Gly, Ser, Thr or absent
  MISC_FEATURE (3)..(6): Xaa is any amino acid
  MISC_FEATURE (10)..(11): Xaa is Ala, Leu, Gly, Ser, Thr or absent
XXXXXXTRPXX

SEQ ID NO: 3 - protein, 11 aa, Artificial Sequence
  7m8 insersertion sequence motif
  MISC_FEATURE (1)..(2): Xaa is Ala, Leu, Gly, Ser, Thr or absent
  MISC_FEATURE (3)..(3): Xaa is Leu, Asn or absent
  MISC_FEATURE (4)..(4): Xaa is Gly, Glu or absent
  MISC_FEATURE (5)..(5): Xaa is Glu or Thr
  MISC_FEATURE (6)..(6): Xaa is Thr or Ile
  MISC_FEATURE (10)..(11): Xaa is Ala, Leu, Gly, Ser, Thr or absent
XXXXXXTRPXX

SEQ ID NO: 4 - protein, 11 aa, Artificial Sequence
  7m8 insersertion sequence motif
  MISC_FEATURE (1)..(2): Xaa is Ala, Leu, Gly, Ser, Thr or absent
  MISC_FEATURE (3)..(3): Xaa is Lys or absent
  MISC_FEATURE (4)..(4): Xaa is Ala or Asp
  MISC_FEATURE (5)..(5): Xaa is Gly or Pro
  MISC_FEATURE (6)..(6): Xaa is Gln or Lys
  MISC_FEATURE (7)..(7): Xaa is Thr or Ala
  MISC_FEATURE (8)..(8): Xaa is Asn or Thr
  MISC_FEATURE (9)..(9): Xaa is Asn or absent
  MISC_FEATURE (10)..(11): Xaa is Ala, Leu, Gly, Ser, Thr or absent
XXXXXXXXXXX

SEQ ID NO: 5 - protein, 11 aa, Artificial Sequence
  7m8 insersertion sequence motif
  MISC_FEATURE (1)..(2): Xaa is Ala, Leu, Gly, Ser, Thr or absent
  MISC_FEATURE (3)..(3): Xaa is a positively charged amino acid or an uncharged amino acid; or is selected from Leu, Asn, Arg, Ala, Ser, and Lys; or absent
  MISC_FEATURE (4)..(4): Xaa is a negatively charged amino acid or an uncharged amino acid; or is selected from Gly, Glu, Ala, Val, Thr, and Asp
  MISC_FEATURE (5)..(5): Xaa is a negatively charged amino acid or an uncharged amino acid; or is selected from Glu, Thr, Gly, Asp, or Pro
  MISC_FEATURE (6)..(6): Xaa is selected from Thr, Ile, Gly, Lys, Asp, and Gln
  MISC_FEATURE (7)..(7): Xaa is a polar amino acid, an alcohol (an amino acid having a free hydroxyl group), or a hydrophobic amino acid; or is selected from Thr, Ser, Val, and Ala
  MISC_FEATURE (8)..(8): Xaa is a positively charged amino acid or an uncharged amino acid; or is selected from Arg, Val, Lys, Pro, Thr, and Asn
  MISC_FEATURE (9)..(9): Xaa is a positively charged amino acid or an uncharged amino acid; or is selected from Pro, Gly, Phe, Asn, and Arg; or is absent
  MISC_FEATURE (10)..(11): Xaa is Ala, Leu, Gly, Ser, Thr or absent
XXXXXXXXXXX

SEQ ID NO: 6 - protein, 7 aa, Artificial Sequence
  Synthesized 7m8 insertion peptide
LGETTRP

SEQ ID NO: 7 - protein, 7 aa, Artificial Sequence
  Synthesized 7m8 insertion peptide
NETITRP

SEQ ID NO: 8 - protein, 7 aa, Artificial Sequence
  Synthesized 7m8 insertion peptide
KAGQANN

SEQ ID NO: 9 - protein, 7 aa, Artificial Sequence
  Synthesized 7m8 insertion peptide
KDPKTTN

SEQ ID NO: 10 - protein, 7 aa, Artificial Sequence
  Synthesized 7m8 insertion peptide
KDTDTTR

SEQ ID NO: 11 - protein, 7 aa, Artificial Sequence
  Synthesized 7m8 insertion peptide
RAGGSVG

SEQ ID NO: 12 - protein, 7 aa, Artificial Sequence
  Synthesized 7m8 insertion peptide
AVDTTKF

SEQ ID NO: 13 - protein, 7 aa, Artificial Sequence
  Synthesized 7m8 insertion peptide
STGKVPN

SEQ ID NO: 14 - protein, 10 aa, Artificial Sequence
  Synthesized 7m8 insertion peptide
LALGETTRPA

SEQ ID NO: 15 - protein, 10 aa, Artificial Sequence
  Synthesized 7m8 insertion peptide
LANETITRPA

SEQ ID NO: 16 - protein, 10 aa, Artificial Sequence
  Synthesized 7m8 insertion peptide
LAKAGQANNA

SEQ ID NO: 17 - protein, 10 aa, Artificial Sequence
  Synthesized 7m8 insertion peptide
LAKDPKTTNA

SEQ ID NO: 18 - protein, 10 aa, Artificial Sequence
  Synthesized 7m8 insertion peptide
LAKDTDTTRA

SEQ ID NO: 19 - protein, 10 aa, Artificial Sequence
  Synthesized 7m8 insertion peptide
LARAGGSVGA

SEQ ID NO: 20 - protein, 10 aa, Artificial Sequence
  Synthesized 7m8 insertion peptide
LAAVDTTKFA

SEQ ID NO: 21 - protein, 10 aa, Artificial Sequence
  Synthesized 7m8 insertion peptide
LASTGKVPNA

SEQ ID NO: 22 - protein, 10 aa, Artificial Sequence
  Synthesized 7m8 insertion peptide
AALGETTRPA

SEQ ID NO: 23 - protein, 10 aa, Artificial Sequence
  Synthesized 7m8 insertion peptide
AANETITRPA

SEQ ID NO: 24 - protein, 10 aa, Artificial Sequence
  Synthesized 7m8 insertion peptide
AAKAGQANNA

SEQ ID NO: 25 - protein, 10 aa, Artificial Sequence
  Synthesized 7m8 insertion peptide
AAKDPKTTNA

SEQ ID NO: 26 - protein, 9 aa, Artificial Sequence
  Synthesized 7m8 insertion peptide
GLGETTRPA

SEQ ID NO: 27 - protein, 9 aa, Artificial Sequence
  Synthesized 7m8 insertion peptide
GNETITRPA

SEQ ID NO: 28 - protein, 9 aa, Artificial Sequence
  Synthesized 7m8 insertion peptide
GKAGQANNA

SEQ ID NO: 29 - protein, 9 aa, Artificial Sequence
  Synthesized 7m8 insertion peptide
GKDPKTTNA

SEQ ID NO: 30 - RNA, 27 nt, Artificial Sequence
  Made in Lab - synthesized aptamer sequence
cgcaaucagugaaugcuuauacauccg

SEQ ID NO: 31 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 32 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL